(19) United States
(12) Patent Application Publication
(10) Pub. No.: US 2001/0012632 A1
(43) Pub. Date: Aug. 9, 2001

(54) Dendritic-like cell/tumor cell hybrids and hybridomas for inducing an anti-tumor response

(76) Inventors: Muriel Moser, Wezembeek-Oppem (BE); Oberdan Leo, Wezembeek-Oppem (BE); Laurence Lespagnard, Bruxelles (BE); Jacques Urbain, Lasne (BE); Catherine Bruyns, Rhode-St-Genese (BE); Catherine Gerard, Rhode-Saint-Genese (BE); Michel Goldman, Brussels (BE); Thierry Velu, Brussels (BE); Fabienne Willems, Linkebeek (BE); Nicole Tasiaux, Bruxelles (BE); Jason Perret, Woulhier-Brauie (BE); AnneMarie Verheyden, Grez-Doiceau (BE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/802,397

(22) Filed: Mar. 9, 2001

Related U.S. Application Data
(62) Division of application No. 09/049,502, filed on Mar. 27, 1998.
Application No. 09/049,502 is a continuation-in-part of application No. 09/025,405, filed on Feb. 18, 1998, now abandoned.
Application No. 09/025,405 is a continuation of application No. 08/625,507, filed on Mar. 29, 1996, now abandoned.
Application No. 08/625,507 is a continuation-in-part of application No. 08/414,480, filed on Mar. 31, 1995, now abandoned.

(51) Int. Cl. A61K 48/00; C12N 5/22; C12N 15/02

(52) U.S. Cl. 435/449; 424/93.21

(57) ABSTRACT

A method produces an anti-tumor response in a mammalian subject in need of anti-tumor treatment. Hybrids of tumor cells with dendritic cells or dendritic-like cells can be administered to the subject. The tumor cell of the hybrid corresponds to the tumor of the subject. Alternatively, autologous immune cells of the subject can be administered in which the immune cells are activated in vitro by co-cultivation with these hybrids.

### RELATED APPLICATIONS

The present application is a divisional of U.S. application No. 09/049,502, filed Mar. 27, 1998, which is a continuation-in-part of application No. 09/025,405, filed Feb. 18, 1998, which is a continuation of application No. 08/625,507, filed Mar. 29, 1996, abandoned, which is a continuation-in-part of application No. 08/414,480, filed Mar. 31, 1995, abandoned.

### FIELD OF THE INVENTION

The invention is in the field of immunotherapy for the treatment of cancer. Specifically, the invention provides hybrids and hybridomas consisting of a fused tumor cell and a dendritic-like cell, preferably a dendritic cell, which is capable of inducing an anti-tumor response in vivo when administered to a subject in need of anti-tumor treatment.

### BACKGROUND OF THE INVENTION

### The Immune Response

The introduction of pathogens such as bacteria, parasites or viruses into a mammal elicits a response contributing to the specific elimination of the foreign organism. Foreign material is referred to as antigen, and the specific response is called the immune response. The immune response starts with the recognition of the antigen by a lymphocyte, proceeds with the elaboration of specific cellular and humoral effectors and ends with the elimination of the antigen by the specific effectors. The specific effectors are essentially T-lymphocytes and antibodies, mediating cellular and humoral immune responses, respectively. The present invention relates to the initiation of a cellular immune response. The initiation of a cellular immune response starts with the recognition of an antigen on the surface of an antigen-presenting cell (APC).

### Antigen Recognition by T-lymphphocytes

Cellular antigen recognition is operated by a subset of lymphocytes called T-lymphocytes. T-lymphocytes include two major functional subsets. They are T-helper lymphocytes (TH), that usually express the CD4 surface marker, and cytotoxic T-lymphocytes (CTL), that usually express the CD8 surface marker. Both T-cell subsets express an antigen receptor that can recognize a given peptide antigen. The peptide needs to be associated with a major histocompatibility molecule (MHC) expressed on the surface of the APC, a phenomenon known as APC restriction. T-cells bearing the CD4 surface marker recognize peptides associated with MHC class II molecules, whereas T-cells bearing the CD8 surface marker recognize peptides associated with MHC class I molecules.

Since the T-cell antigen receptor can only recognize peptides associated with MHC molecules at the surface of an APC, cellular proteins need to be processed into such peptides and transported with MHC molecules to the cell surface. This is referred to as antigen processing. Exogenous proteins, phagocytosed by the APC, are broken down into peptides that are transported on MHC class II molecules to the cell surface, where they can be recognized by CD4⁺ T-cells. In contrast, endogenous proteins, synthesized by the APC, are also broken down into peptides, but the latter are transported on MHC class I molecules to the cell surface, where they can be recognized by CD8⁺ T-cells.

When a T-cell binds through its antigen receptor to its cognate peptide-MHC complex on an APC, the binding generates a first signal from the T-cell membrane towards its nucleus. However, this first signal is insufficient to activate the T-cell, at least as measured by the induction of IL-2 synthesis and secretion. Activation only occurs if a second signal or costimulatory signal is generated by the binding of other APC surface molecules to their appropriate receptors on the T-cell surface. The best known costimulatory molecules identified to date on APC are B7-1 (Razi-Wolf et al., Proc. Natl. Acad. Sci. USA 90, pp. 11182-1186 (1993)) and B7-2 (Hathcock et al., Science 262, pp. 905-907 (1993)); both bind to the CD28/CTLA4 counter-receptor on T-lymphocytes. The capacity to present peptide antigens together with costimulatory molecules in such a way as to activate T-cells is hereafter referred as to as antigen presentation. Only APCs have the capacity to present antigen to CD4⁺ (predominantly TH) and CD8⁺ (predominantly CTL) T-cells, leading to the development of humoral and cellular immune responses.

### T-lymphocyte Activation By Antigen-presenting Cells

APCs are heterogeneous in their cell lineage and functional performance. They include distinct cell types such as B-lymphocytes-, T-lymphocytes, monocytes/macrophages and dendritic cells from myeloid origin. All these cells are bone marrow-derived cells, that need to mature and to be activated in order to function efficiently as APCs.

The functional performances of APCs rely critically upon the nature and state of maturation of the cells included in purified or enriched APC preparations. The latter vary with the tissue of origin and method of purification. In an operational way, we call dendritic-like cells (DLCs) or dendritic cells all non-B cells present in purified or enriched preparations of dendritic cells. These cells all share some morphological, physical or biochemical characteristics with dendritic cells, leading to their co-purification with dendritic cells. Therefore, the term DLCs refers hereafter preferably but not only to dendritic cells (DC) of myeloid origin, but also to monocytes, T-lymphocytes and other non-B cells present in enriched or purified dendritic-like cell preparations. In mice, the spleen is very often used as a source of DLCs (reviewed by Steinman, Annu. Rev. Immunol. 9, pp. 271-29G (1991)). However, mouse DLCs or DCs have also been generated by in vitro culture from bone marrow progenitors in the presence of cytokines (Inaba et al., J. Exp. Med. 176, pp. 1693-1702 (1992)). In humans, blood or bone marrow are the usual sources of DLCs and DCs that are used either immediately or more often after culture in the presence of cytokines. Several protocols of purification and in vitro culture have been published (reviewed in Young and Inaba, J. Exp. Med. 183, pp. 7-11 (1996)), and patent applications have been filed for some of them (WO93/20185 by Steinman R., Inaba K. and Schuler G., WO93/20186 by Banchereau J. and Caux C., WO94/02156 by Engelman E., Markowicz S. and Metha A., WO95/28479 by Brugger W. and colleagues of Mertelsmann r.).

### T-lymphocytes Activation by Tumor Cells

There is increasing evidence that tumor cells do not usually function as APCs (reviewed by Young and Inaba, J. Exp. Med. 183, pp. 7-11 (1996)). Although some tumor cells are capable of delivering an antigen-specific signal to T-cells, they may not provide the costimulatory signals which are necessary for the full activation of T-cells and thereby fall to induce an efficient anti-tumor immune response. In order to compensate for this inefficient induction of an anti-tumor immune response, different approaches have been tried in experimental animals (reviewed by Grabbe et al., Immunology Today 16, pp. 117-121 (1995)).

In one such approach, tumor cells were genetically engineered to express one or more molecules known to be involved in antigen presentation on APC. To date, efficient in vivo results from this approach were obtained with tumor cells co-expressing MHC class I, MHC class II and B7-1 molecules, suggesting that the successful immunotherapy was linked to the activation of both CD4⁺ and CD8⁺ T-cells. For example, Basker et al. (J. Exp. Med. 181, pp. 619-629 (1995) engineered mouse fibrosarcoma cells, that naturally express MHC class I molecules, to express in addition MHC class II molecules and B7-1 molecules; the injection of these modified tumor cells was sufficient to cure stngeneic mice carryiing large established tumors. It should be noted that tumor cells expressing MHC class I molecules but not MHC class II molecules and transduced with the B7-1 costimulator also induced an in vivo anti-tumor immune response, and that the latter depended utpon the activation of CD8⁺, but not CD4⁺ T-cells (Ramarathinam et al., J. Exp. Med 179, pp. 1205-1214 (1994)). The disadvantage of this approach lies in the genetic engineering of the tumor cells, a technique that usually involves the use of viral vectors for efficient gene transfer. Viral vectors are not totally safe for the treatment of human patients. The main reason is that they can recombine both in vitro and in vivo, which may lead to the production of novel wild type viruses of unpredictable pathogenicity. This limitation stimulated the development of alternative methods of efficient gene transfer, such as the one recently described by Birnstiel et al. (WO94/21808)

In another approach, APCs were loaded with a source if tumor antigens. Amongst the APCs tested for such a purpose, DLCs appeared to be the most efficient. To date, it is clear that DLCs pulsed with tumor cell lysates (Knight et al., Proc. Natl. Acad, Sci. USA 82, pp. 4495-4497 (1985)), with a purified tumor-associated protein (Flamand et al., Eur. J. Immunol. 24, pp. 605-610 (1994), Paglia et al., J. Exp. Med. 183, pp. 317-322 (1996)) or with tumor-associated peptides (Ossevoort et al., J. Immunotherapy 18, pp. 86-94 (1995), Mayordomo et al., Nature Medicine 1, pp. 1297-1302 (1995)) can efficiently induce an anti-tumor response in vivo. There are, however, disadvantages to this approach. Tumor cell lysates or fractions thereof are relatively easy to prepare, but the loading of DLCs with such crude preparation could, at least theoretically, induce adverse auto-immune reactions in the host. Similar secondary effects could be induced by DLCs loaded with all the peptides eluted from tumor cells, as described by Zitvogel et al. (J. Exp. Med 183, pp. 87-97 (1996)). The latter risk is reduced by pulsing DLCs with purified, tumor-specific antigens or peptides. However, there are very few known tumor-specific antigens, and in addition, their production and purification are both labor-intensive and expensive.

In a recent approach, a tumor cell and one sort of APC, namely a B-lymphocyte, were united into a single cell by somatic cell fusion (Guo et al., Science 263, pp. 518-520 (1994)). Guo et al. fused a rat hepatoma cell line with in vivo activated B-lymphocytes, and showed that some of the resulting B-cell/tumor cell hybridomas induced tumor-resistance in syngeneic rats and also cured the animals of a small pre-established tumor. The selected hybridomas expressed MHC class II restriction elements and B7 costimulatory molecules, which strongly suggested that the immunotherapy worked through the activation of CD4⁺ TH cells. When compared to the two previous approaches, this third approach has the general advantages of somatic cell fusion, namely, it brings together not only the known tumor antigens and known costimulators of activated B-cells, but possibly some as yet unknown molecules carrying out these functions. When compared to the genetic engineering of tumor cells, this cellular engineering does not require the identification of the genes encoding costimulatory molecules, nor their transfer into tumor cells. Similarly, when compared to the pulsing of APC with purified tumor-specific antigens, somatic cell fusion does not require the identification of genes encoding tumor-specific antigens, nor the production and purification of the corresponding recombinant proteins. However, in its present description, this approach is inapplicable to human cancer patients, because it involves the use of in vivo-activated B-cells as fusion partners of the tumor cells. In vivo-activated B-cells cells were recovered from the spleen fourteen days after immunization with soluble antigen in complete Freund's adjuvant, which cannot be used in humans. In addition, if immunizations are done without Freund's adjuvant, the outcome of an in vivo activation of B-cells remains unpredictable in individual animals, and it is expected to be unpredictable in individual human patients. Finally, he selection of the hybridomas is quite labor-intensive. It required the preparation, absorption and characterization of tumor-specific polyclonal antisera, that were used to select the cells expressing surface markers of the tumor parent; this first selection was then followed by a second selection of cells expressing surface markers of the in vivo-activated B-cell parent.

There is evidence that the failure of the immune system in controlling tumor growth may be due to a deficient costimulation rather than the lack of antigenic peptides presented in the context of self MHC. Indeed, many spontaneous or experimental tumors, in rodents and humans, express specific antigens that are potential targets of a specific immune response. In particular, the methylcholanthrene-induced P815 mastocytoma has been showed to display at least five antigens that are target of cytotoxic T-cells. However, injection of P815 cells in immunocompetent syngeneic hosts results in an initial period of growth that is followed by partial regression and subsequent escape of tumor cells, leading to death (Uyttenhove et al. (1983)). The partial rejection phase suggests that a transient equilibrium is reached between the tumor-specific immune response and the growing tumor, which is disrupted in favor of tumor cells.

It has been showed that optimal activation of T-cells required two signals provided by the antigen-presenting-cell (APC) the antigenic signal and the costimulatory signal which can be provided by the binding of B7-1 or B-2 molecules on the CD28 counter-receptor expressed T-lymphocytes. Recognition of the antigen/MHC complexes in the absence of-costimulation not only fails to activate the cells, but may lead to a state called anergy, in which the T-cell becomes refractory to activation. Importantly, it has been showed that antigen-specific and costimulatory signals were best presented simultaneously on the same cell. Collectively, these observations have led to the hypothesis that a limitation of the tumor-specific immune response may be at the level of antigen presentation, since most tumors do not express B7-1 or B7-2 molecules.

Among the APCs, DCs are considered as the natural adjuvant of the primary immune response in vitro and in vivo (Steinman (1991)). Their unique ability to sensitize naive T-lymphocytes correlates with distinctive features, which include elevated expression of MHC and costimulatory molecules (Inaba et al. (1994)), specialized function over time (Romani et al. (1989)) and migratory properties (De Smedt et al. (1996), Steinman et al. (1997)).

What is really needed is a method to harness the ability of DLCs, preferably DCs, to elicit an anti-tumor response, so that the immune system of a subject can mount a rejection of the tumor cells. In addition, this method should be transposable to human cancer patients.

### SUMMARY OF THE INVENTION

The present invention provides dendritic-like cells (DLC) /tumor cell and dendritic cells (DC) /tumor cell hybridomas and a plurality of dendritic-like cells (DLC)/tumor cell hybrids for use in the treatment of cancers. The hybridomas and hybrids of the invention are capable of inducing an anti-tumor response when administered to the subject, in vivo. Preferably, said dendritic cell (DC) of the hybridoma is a bone marrow derived dendritic cell (DC).

A dendritic-like cell (DLC)/tumor cell hybridoma or a dendritic cell (DC)/tumor cell hybridoma of the invention is produced by first providing a sample of the specific tumor against which an immune response is needed.

In one embodiment of the invention, an immortal cell line is derived from the tumor sample, and then the tumor cells are fused with DLCs or DCs. Preferably, autologous DLCs or DCs from the subject are used, but matched HLA-compatible DLCs or DCs may also be used as fusion partners. Once the DLCs or DCs are fused with the tumor cells, selection is carried out. In this embodiment, hybridomas which exhibit DLCs or DCs characteristics are selected, their immortality being necessarily contributed by fusion with the tumor cell.

In a second embodiment of the invention, an established immortal human tumor cell line is provided which expresses at least one of the tumor-associated antigens of the patient's tumor cells. Cells from the tumor cell line are fused with autologous or HLA-compatible allogeneic DLCs or DCs to form hybridomas which are then selected for retention of DLC or DC characteristics.

In a third embodiment of the invention, an immortal DLC or DC line is established, and then DLCs or DCs of this line are fused with the patient's tumor cells from primary culture. The resulting hybridomas are selected for retention of DLC or DC characteristics as well as expression of at least one tumor-associated antigen of the patient's tumor cells.

In other embodiments of the invention, tumor cells are fused with DLCs, and the resulting plurality of hybrids is used directly for treatment, without selection.

The DLC/tumor cell or DC/tumor cell hybridomas, or plurality of hybrids, are administered to the subject to induce an immune response against residual tumor cells in the subject's circulation or organs or to prevent the growth of said established tumor. Alternatively, the hybridoma or plurality of hybrids is co-cultivated in vitro with immune cells from the subject in order to activate against the tumor cell; the activated immune cells are then returned (administered) to the subject.

### Definitions

Herein, the term “dendritic-like cell (DLC)” is an operational term referring to a non-B cell present in preparations of purified or enriched dendritic cells. DLCs can be dendritic cells of myeloid (*) origin, monocytes, cells intermediate between dendritic cells and monocytes, T-cells or other non-B cells present in the preparation.(*):or lymphoid.

Herein, the term “dendritic cell (DC)” refers to an isolated dendritic cell or its dendritic progenitor, being preferably a bone marrow derived dendritic cell, preferably obtained by the procedure derived from the protocol of Inaba et al. (1992) and Zorina et al. (1994) and described in the Example 12.

Herein, the term “DLC/tumor cell hybrid” is defined as a fused cell which exhibits characteristics of both a DLC and the specific tumor cell of interest. Since a DLC may be a dendritic cell, a monocyte, a T-lymphocyte or another non-B cell co-purifying with dendritic cells, DLC/tumor cell hybrids may include hybrids with different phenotypic characteristics reflecting these different cell fusion partners. A plurality of DLC/tumor cell hybrids is capable of eliciting an immune response, either in vivo or in vitro, against the tumor fusion partner which makes up part of the genome of the hybrids. This capacity is not inhibited by the presence of unfused DLCs, DLC lines or unfused tumor cells or tumor cell lines.

Herein, the term “DLC or DC/tumor cell hybridoma” is defined as an immortal hybrid cell line, which exhibits characteristics of both a DLC or a DC and the specific tumor cell of interest. Since a DLC may be a dendritic cell, a monocyte, a T-lymphocyte, and other non-B cells co-purifying with dendritic cells, DLC/tumor cell hybridomas may exhibit phenotypic characteristics of any of these cell lines. For instance, in examples below, 2 murine DLC/tumor cell hybridomas exhibited T-cell lineage characteristics, whereas 1 human DLC/tumor cell hybridoma was likely from monocytic origin. More importantly, a DLC or DC/tumor cell hybridoma is capable of eliciting an immune response, either in vivo or in vitro, against the tumor fusion partner which makes up part of the genome of the hybridoma.

Herein, the term “anti-tumor response in vivo” refers to the in vivo induction of immune effectors that confer resistance to a subsequent challenge with tumor cells, contribute to the rejection of pre-existing tumor cells and/or prevent or reduce the growth of tumors made of said tumor cells. In Example 5B, these immune effectors include cytotoxic T-lymphocytes that were detected by submitting the spleen cells of the immunized animals to an in vitro assay. In human subjects, appropriate non-invasive measures can be used for demonstrating the presence of anti-tumor immune effectors. However, the clinical course of the tumor, monitored by imaging techniques and the survival of the patient, will be the prime criterion for the evaluation of the immunotherapy. In the example 12, the immune effectors include the generation and proliferation of cells displaying cytotoxic activity to tumoral cells as well as the development of IL-2 secreting cells.

Herein, the term “anti-tumor response in vitro” refers to the in vitro activation of autologous immune cells into anti-tumor immune effectors. The latter will contribute to the rejection of the pre-existing tumor cells when infused into the patient. The secretion of IL-2 by the murine T-DLC/tumor cell hybridomas (Example 6) and the secretion of GM-CSF by the human (presumed monocytic) DLC/tumor cell hybridoma may contribute to such in vitro and in vivo activation of anti-tumor immune cells.

Herein, the term “TDLC or DC characteristics” shared by the hybridoma of the invention refers to DLC or DC morphology, the expression of DLC or DC surface markers, the expression of DLC or DC genetic markers and/or the activation of immune cells.

Herein, the term “DLC or DC morphology” refers to a typical image observed by scanning electron microscopy. The images of the DLC or DC/tumor cell hybridoma are compared to those of the parent tumor cell, DC and DLC. At first glance, to one skilled in the art, it is clear that the hybridoma resembles the DLC or DC more than the tumor cell. Upon analysis, DLCs or DCs have irregular shapes, due to the presence of clearly-visible, flat cytoplasmic extensions like pseudopodia and veils. Hybridomas with such similar cytoplasmic extensions can be recognized as having a dendritic-like cell morphology, as illustrated in FIG. 1 (see Example 4). These data are also consistent with the possibility that other embodiments of the present invention may express these or other DLC or DC morphological traits, since the DLC morphology of a DLC or DC/tumor cell hybridoma is expected to mirror the particular morphology of the DLC used as a fusion partner.

Herein, the term “expression of DLC or DC surface markers” refers to the expression of markers restricted to the DLCs or DCs used for fusion. These markers include T-cell activating molecules and other molecules. T-cell activating molecules are expressed on activated APCs; they include mainly MHC class I and class II restricting elements, as well as the family of B7 costimulatory molecules; the latter bind to the CD28/CTLA4 counter-receptor on T-cells. Other DLC surface markers include, for example, CD1a for human myeloid dendritic cells, CD14 for monocytes, and the TCR/CD3 complex for T-cells. It is shown in Example 4B (Table 1 and FIG. 2) that the HY41 and HY62 hybridomas express MHC class I molecules and the TCR/CD3 complex, but neither MHC class II molecules, nor B7 costimulators. When such T-cell activating molecules are not expressed on resting hybridomas, they can sometimes be induced by exposure to cytokines or other activating agents; Example 10B illustrates such an induced expression of HLA-DR on a human DLC/tumor cell hybridoma.

Herein, the term “tumor-associated antigen” refers to a peptide derived from a protein expressed by a tumor cell which, when expressed by the hybridoma of the invention, will enable the hybridoma to elicit a tumor-specific response in vivo and/or in vitro. It also refers, by extension, to the proteins from which the antigenic peptides are derived, and to the genes encoding the antigenic proteins.

Herein, the term “activation of immune cells in vivo” refers to the immune rejection of a residual tumor, as measured by its reduction in size and by the survival of the patient, as shown for mice in Example 5C or Example 12. In vitro correlates of this in vivo state of immunity include for example the detection of blood or tissue immune cells able to kill the patient's own tumor cells in vitro. In experimental animals, the quoted expression also refers to the immune rejection of the living hybridoma, to the immune resistance to a subsequent inoculation of tumor cells, and to the presence of tumor-specific cytolytic effector cells in the lymphoid organs of the tumor-resistant animals, as shown in Example 5.

Herein, the term “activation of immune cells in vitro” refers for example to a mixed lymphocyte-tumor cell reaction, wherein the dendritic cell/tumor cell hybridoma (“the tumor cell”) stimulates one of the following reactions by allogeneic T-cells (“the lymphocyte”) : (1) T-cell proliferation, as measured by tritiated thymidine incorporation; (2) T-cell secretion of cytokines including for example IL-2, interferon-gamma and others, as measured by ELISA, bioassay, or reverse transcription polymerase chain reaction; (3) T-cell-mediated tumor cell lysis, as measured by chromium release assay. This term may also refer to the activation of other immune cells, like monocytes and natural killer cells, and can be measured, for example, by cytokine release or cytotoxic cell assays.

### BRIEF DESCRIPTION OF THE DRAWINGS

FIG. 1

Scanning electron microscopy of parent cells and of two murine DLC/tumor cell hybridomas (×4,000). The figure illustrates the “tumor-like” and “dendritic-like” characteristics of two DLC/tumor cell hybridomas. Hybridoma HY1 (FIG. 1c) resembles more the parent P815* tumor cell (FIG. 1a) than the parent dendritic cell (FIG. 1b), whereas hybridoma HY41 (FIG. 1d) resembles more the dendritic cell (FIG. 1b) than the P815* tumor cell (FIG. 1a). It is the “dendritic-like” hybridoma HY41 that was selected for in vivo experiments.

FIGS. 2a-e

FACS analysis of DLC/tumor cell hybridomas HY41 and HY62, showing the expression of CD3 and the TCR V-β8 domain by the CD3-positive subclones (HY41 CD3⁺ and HY62 CD3⁺); the CD3-negative subclones of these hybridomas (HY41 CD3⁻ and HY62 CD3⁻) as well as the parent P815* tumor cells fail to express the TCR V-β8 domain.

FIG. 3

Ethidium bromide-stained gel electrophoresis of Polymerase Chain Reaction products obtained with mouse genomic DNA, using TCR Vβ8 and C(primers. A rearranged TCR(gene fragment was amplified from genomic DNA of a mouse T-cell hybridoma (T), as well as from the HY41 (41) and HY62 (62) DLC/tumor cell hybridomas; no rearranged TCR(fragment was amplified from DNA of P815* tumor cells (P) and spleen cells (S), used as negative controls.

FIG. 4

Survival curves of immunocompetent and immunocompromised (i.e. irradiated) DBA/2 mice after ip inoculation with 5× 10⁵ syngeneic hybridoma cells HY41 or with the same number of parental P815* tumor cells. Key : ∘P815 in normal mice (n=10) ● P815 in irradiated mice (n=10) Δ HY 41 in normal mice (n=12) ▴ HY 41 in irradiated mice (n=10)

This figure shows that the “dendritic-like” hybridoma HY41 was rejected by 75% (9/12) of the immunocompetent mice, while the parent tumor was rejected, in this particular experiment, by 20% (2/10) of the animals. This difference in survival was not due to a difference in tumorigenicity, since both cell lines killed all (10/10) immunocompromised animals within four weeks of inoculation.

FIG. 5

Survival curves of naive and HY41-treated DBA/2 mice after ip inoculation with 5×10⁵ syngeneic P815* tumor cells. Y-axis=survival (%) X-axis=weeks after inoculation. Key: ∘ normal mice (n=9) ∇ “HY41”—treated mice (n=9)

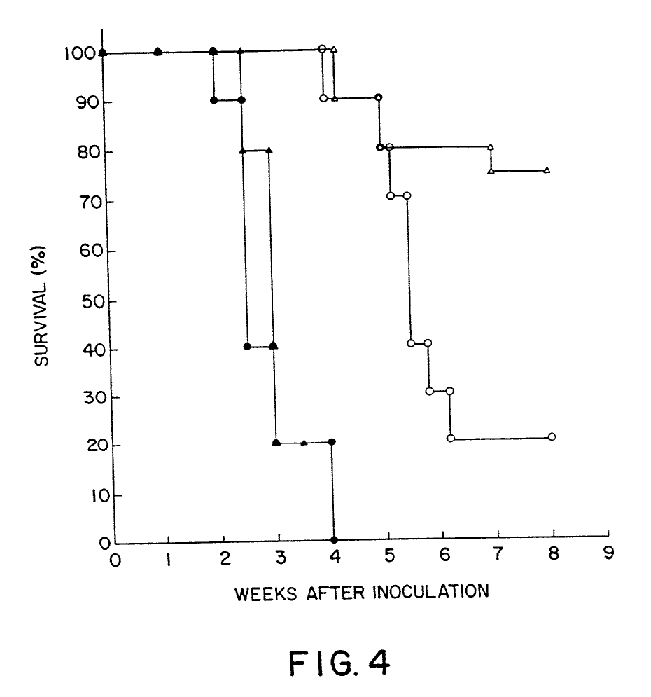

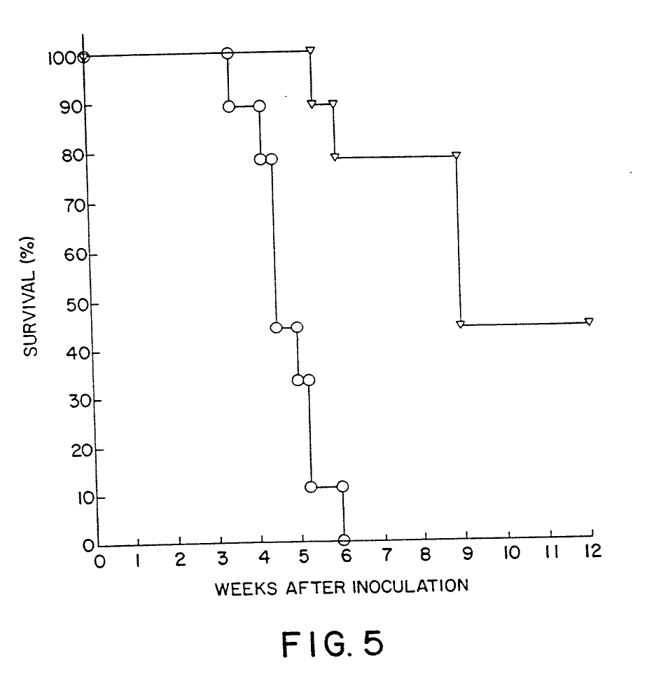

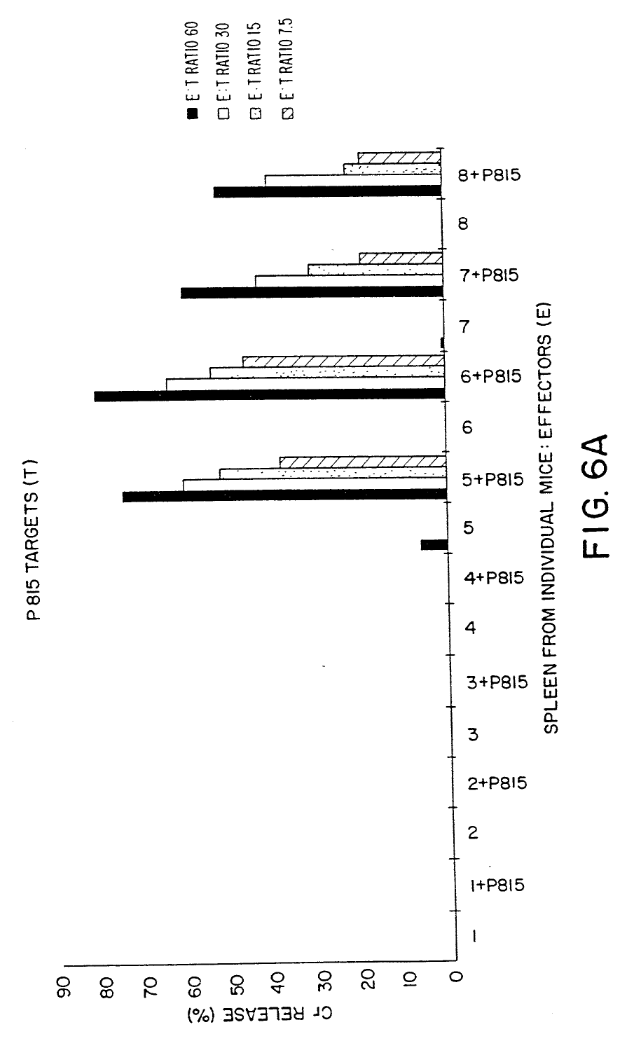

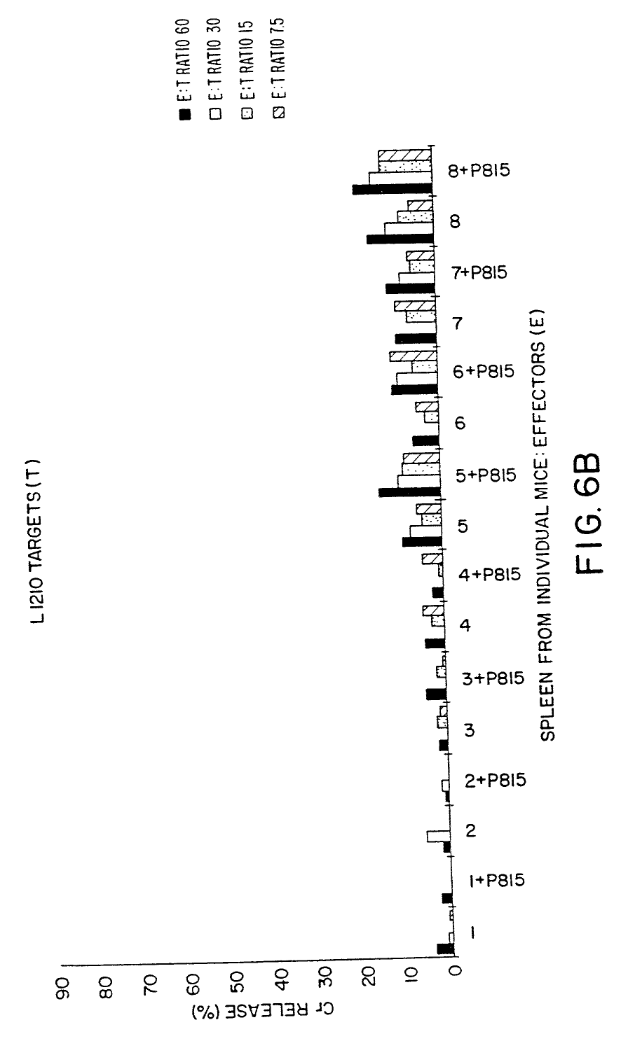

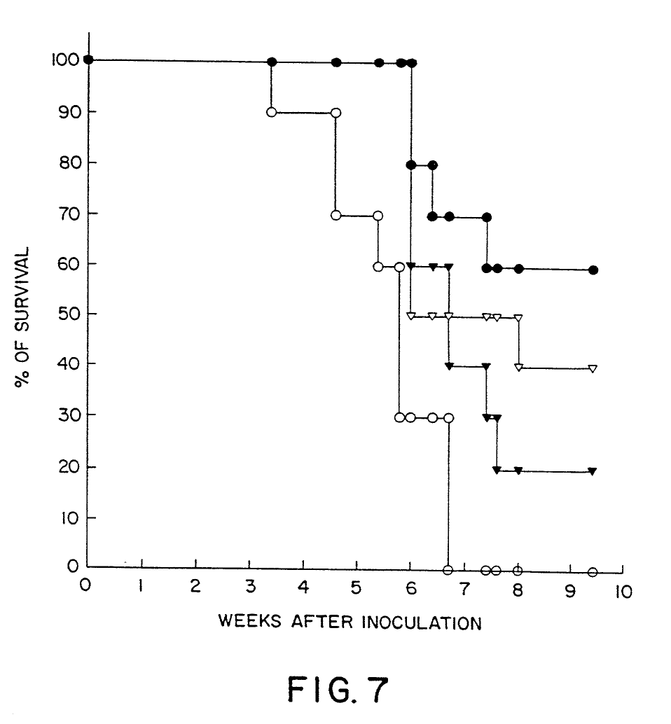

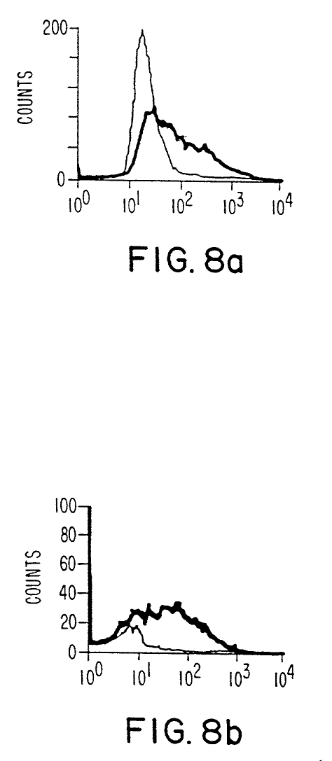

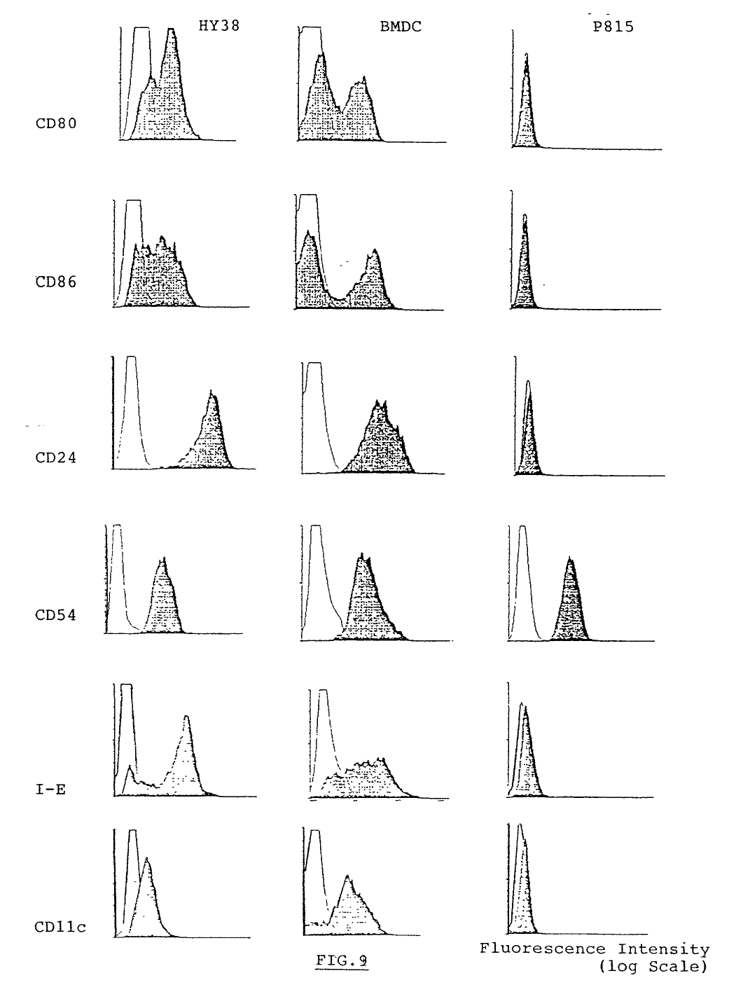

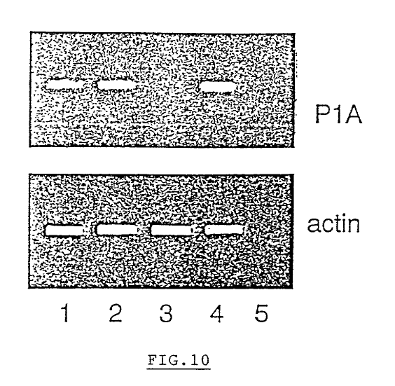

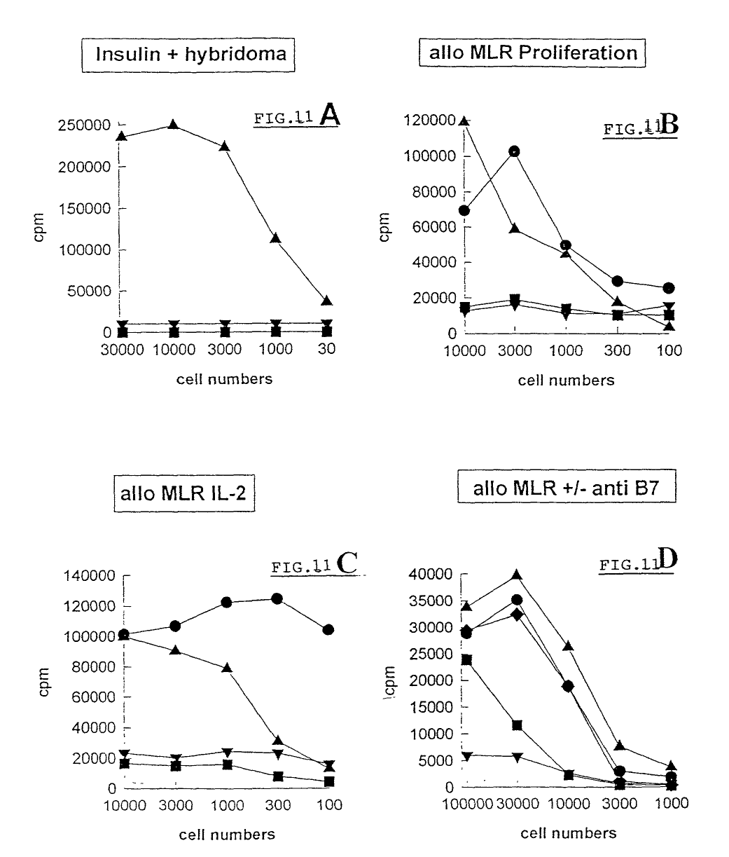

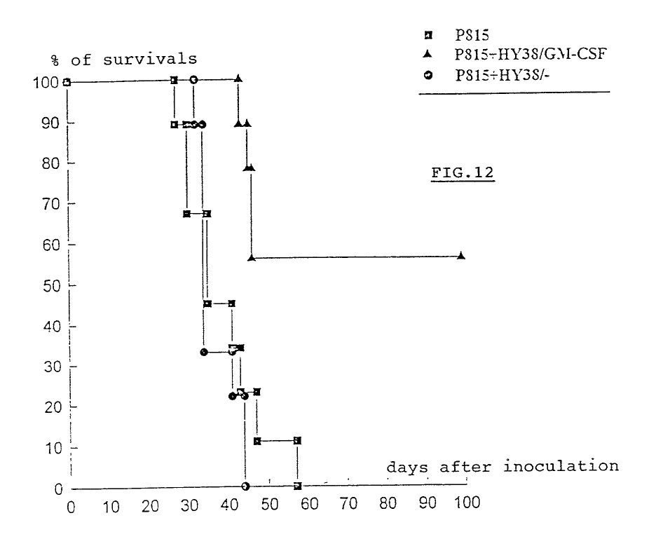

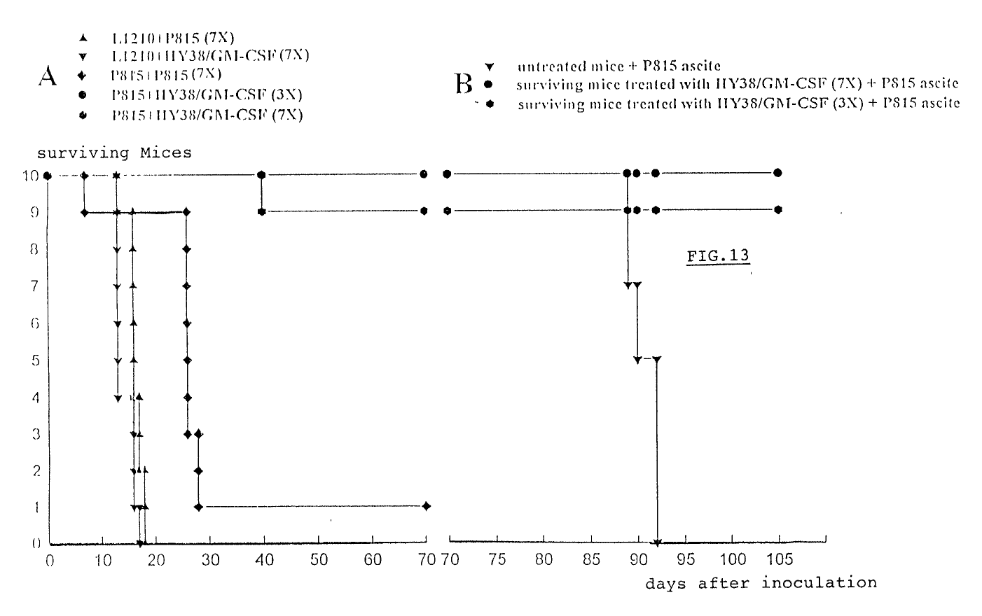

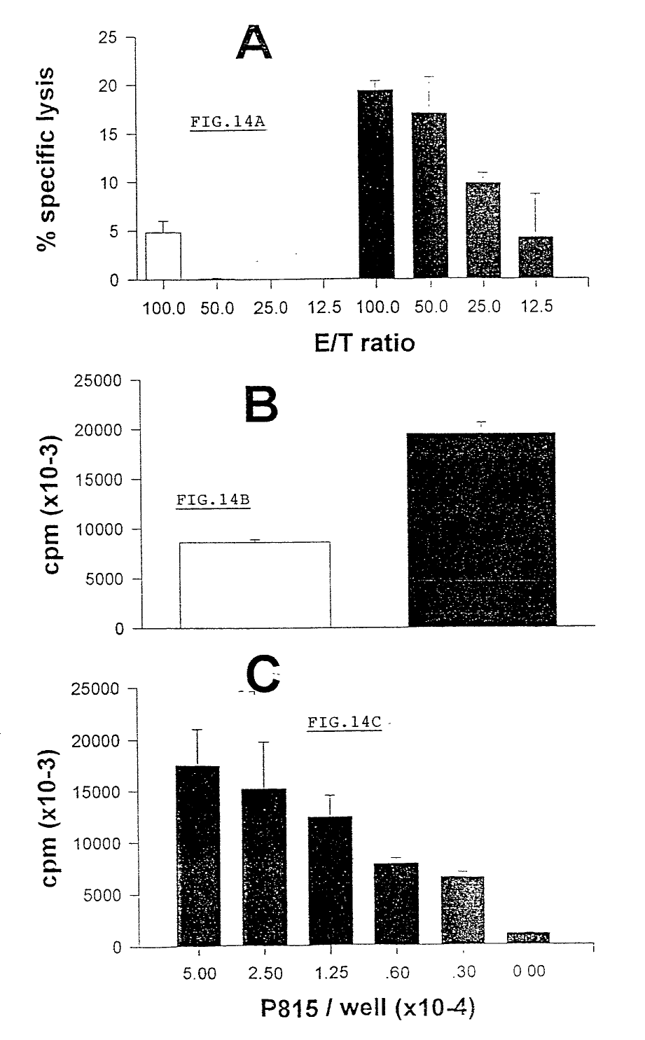

This figure shows that the nine HY41-survivors (see FIG. 4) became at least partially resistant to a lethal challenge with the parental P815* tumor cells, and that 4/9 of these animals showed complete tumor resistance for at least three months.

FIG. 6

P 815 Targets (T). Chromium release assay on P815* and L1210 target cells with spleen cells from individual mice. Y-axis=Cr release (%) X-axis=spleen from individual mice:effectors (E). This figure shows that the spleen cells of the four P815*— resistant mice (see FIG. 5; individual mice nrs 5 - 8 in FIG. 6), contain a strong cytolytic activity directed against P815* cells (FIG. 6A) but not against the irrelevant (but MHC class I-matched) L1210 tumor cells (FIG. 6B). In contrast, the spleen cells of the four naive animals (mice nr 1-4) do not show any detectable cytolytic activity against P815* cells (FIG. 6A). The spleen cells from individual mice (1 - 8) were cultured in vitro for five days either in the absence (x) or in the presence of P815* stimulator cells (x+P815). Thereafter, they were used as effector cells on chromium-labeled target cells, at different effector:target (E:T) ratios.

FIG. 7

Survival of mice bearing an established tumor P815*. Y-axis=% of survival. X-axis=weeks after inoculation. Key: ∘ untreated mice (n=10) ● HY41—treated mice (n=10) ∇ HY62—treated mice (n=10) ▾ P815—treated mice (n=10)

Survival curves of tumor-inoculated mice treated with irradiated HY41 or HY62 hybridoma cells. All mice were inoculated ip with 2×10⁵ P815* tumor cells on day 0. The figure shows that 2 months after tumor inoculation, 6/10 and 4/10 animals treated by 4 weekly ip injections of irradiated HY41 and HY62 hybridoma cells, respectively, were alive and tumor-free. In contrast, none (0/10) of the untreated animals and only 2/10 animals treated with irradiated P815* tumor cells were alive at that same time.

FIG. 8

FACS analysis showing HLA-DR expression in human F3BG10 DLC/tumor cell hybridoma (FIG. 8a) and in its subclone F3BG10-H12 (FIG. 8b) before and after incubation with interferon γ. Before incubation with the cytokine, labeling by the anti-HLA-DR mAb (thinner line) was identical to the labeling by the isotope-matched control mAb (not shown). After 24 hours incubation with interferon γ, around 40% of the F3GG10 hybridoma cells and over 90% of the H12 subclone cells were specifically labeled by the anti-HLA-DR mAb (thicker line).

FIG. 9

Hybrid cells express B7-1 (CD80), B7-2 (CD86), HSA (CD24), ICAM-1 (CD54), I-E, and CD11c. GM-CSF-treated hybrid cells, bone marrow-derived DC and P815 cells were stained with fluoresceinated monoclonal antibodies. Solid areas show cells stained with the corresponding antibodies; open areas show unstained cells.

FIG. 10

Expression of mRNA specific for P815-associated antigen P1A. Primers specific for the PlA and actin sequences were used to amplify RNA isolated from hybrid cells cultured with (lane 2) or without (lane 1) GM-CSF, bone marrow-derived DC (lane 3) and P815 cells (lane 4). Negative control (no DNA) is shown in lane 5. The PCR products were analyzed by 3% agarose gel electrophoresis and visualized by ethidium bromide staining.

FIG. 11

Hybrid cells process exogenous protein and sensitize allogeneic naive T-lymphocytes in vitro. (A) Various numbers of P815 (▾) cells or hybrid cells, cultured with (▴) or without GM-CSF (▪), were cultured in the presence of 5×10⁴ T-cell hybridoma B8P4.1C3 and 200 μg/ml pork insulin. IL-2 was quantified from the 24 h culture supernatant using a standard bioassay using an IL-2-dependent, IL-4 insensitive subclone of the CTL.L line. (B, C). Various numbers of γ-irradiated P815 cells (▾), hybrid cells treated with (▴) or without (▪) GM-CSF, or bone marrow-derived DC (n) were cultured with 2×10⁵ T-cells from CBA mice. (B) Proliferation was assessed by adding ³H-thymidine for the last 16 h of a 4-day culture. (C) IL-2 secretion was quantified from the 48 h culture supernatant, as described above. (D) Various numbers of γ-irradiated, GM-CSF-treated hybrid cells were cultured with 2×10⁵ T-lymphocytes from CBA mice in the absence (♦) or in the presence of anti-B7-1 (●), anti-B7-2 (▪) or both (▾) mabs or a combination of isotype-matched control antibodies (▴). IL-2 secretion was quantified from the 48 h supernatant as described above.

FIG. 12

Repeated injections of HY38 cultured with GM-CSF prevent the growth of pre-established P815 mastocytoma. 2×10⁵ P815 cells were inoculated intraperitoneally into 3 groups of 10 DBA/2 mice (day 0). Two groups were further injected intraperitoneally on day 3, 8, 13, 18, 23, 28 and 33, with 2×10⁶ γ-irradiated (15000 rads) HY38 cultured with or without GM-CSF.

FIG. 13

Three injections of hybrid cells induce tumor-specific long-term protection.

(a) 2 groups of 10 DBA/2 mice were inoculated intraperitoneally with 2×10⁴ L1210 cells, and 3 groups were injected with 2×10⁵ P815 cells. The mice were further treated with 3 (3×) or 7 (7×) injections of 2× 10⁶ irradiated P815 or hybrid cells every 5 days starting on day 3.

(b) Surviving mice (19) and control animals (10) were inoculated intraperitoneally with 2×10⁵ P815 cells harvested from ascitic fluid of irradiated mice injected with P815 cells.

FIG. 14

Characterization of the immune response of surviving mice. (A, B) Splenocytes from surviving mice (pool of five), injected with P815 and irradiated hybrid cells and challenged with P815, were cultured in medium alone (open bars) or with irradiated P815 cells (solid bars).

(a) The effector cells were tested 5 days later for their lytic activity on P815. Results are expressed as percent specific lysis at indicated effector/target ratios.

(b) IL-2 was measured in culture supernatants collected after 24 h of culture, as described above.

(c) Peritoneal exudate cells were harvested from the same animals and cultured with various numbers of irradiated P815. The supernatants were collected after 48 h of culture and assayed for IL-2 content. Data are expressed as mean of triplicates ±SD (95% confidence). The experiments are repeated three times (for spleen cells) and four times (for peritoneal exudate cells) with similar results.

### DETAILED DESCRIPTION OF THE INVENTION

The present invention provides DLC or DC/tumor cell hybrids and hybridomas for activating anti-tumor responses. Although the specific procedures and methods described herein are first exemplified using a DBA/2 mouse mastocytoma cell line and DLCs or DCs isolated from syngeneic spleen or from bone marrow progenitors, they are merely illustrative for the practice of the invention. Analogous procedures and techniques are applicable for the treatment of human subjects, as thereafter exemplified using a human osteosarcoma cell line and blood-derived DLCs or DCs. Therefore, DLC or DC/tumor cell hybrids and hybridomas could be used to immunize human patients against their cancer. Procedures applicable to the treatment of a human subject would involve the following steps:

A sample is provided of the tumor against which an immune response is needed. Such a sample can be obtained when the primary tumor and/or its metastases are removed by surgery, as practised for example for cancers of the breast, prostate, colon, and skin. When the treatment of the cancer involves chemotherapy and/or radiotherapy rather than surgery, as practised for example for small cell lung cancer, lymphomas and leukemias, a sample of the tumor can be obtained from a metastatic site, either before treatment or after relapse. Examples of easily-accessible tumor sampling sites are the peripheral blood, bone marrow, peritoneal and pleural effusions, lymph nodes and skin.

Tumor cells can be separated from blood or bone marrow samples, for instance, by a combination of physical, enzymatic and immunological methods. Contaminating red blood cells can be removed by osmotic lysis. Tumor cells can be concentrated by density centrifugation. Tumor cells can be separated from other cells by binding antigen on the tumor cell surface to antibody-coupled magnetic beads, which are then separated from the biological fluid by means of magnets.

In negative cell selection, which may be performed prior to positive cell selection, antibodies bind to antigens that are expressed on contaminating cells, and used to deplete the biological fluids of non-tumor cells. In positive cell selection, antibodies bind to tumor-associated antigens, and this binding is used to separate tumor cells from the biological fluids.

When tumor cells are separated by means of antibody-coupled magnetic beads, cells can be released from the beads by digestion of the antigen/antibody binding sites with chymopapain or by other means. The resulting separated tumor cells can re-express the tumor-associated antigen after a short time in culture. The tumor cells are expected to contribute genes encoding known and unknown tumor-associated antigens to the hybridoma of the invention.

Tumor cells can also be separated from solid tissue samples, using a combination of physical, enzymatic and immunological methods. Macroscopic peri-tumoral stromal tissue can be removed by dissection prior to reduction of the tumor to a cell suspension. Density centrifugations and antibody-mediated separations can then be performed on the cell suspension as described above.

The purified tumor cells are then prepared for cell fusion. Three types of tumor partners can be prepared: (i) primary cultured tumor cells, (ii) immortal tumor cells, and (iii) drug-sensitive immortal tumor cells. Primary cultured tumor cells are purified tumor cells which have been cultured for a limited period of time in the presence of appropriate growth factors. Immortal tumor cells are permanent cell lines derived from these primary cultured tumor cells; such permanent cell lines can be obtained, for instance, after culturing the primary tumor cells for longer periods of time in the presence of appropriate growth factors, or by transducing the primary tumor cells with immortalizing genes.

Finally, drug-sensitive immortal tumor cells are permanent cell lines derived from spontaneous mutants of immortal tumor cells; these mutants are selected by culturing the immortal tumor cells in the presence of an appropriate drug. These drug-sensitive immortal tumor cells die when they are exposed to the drug to which they are sensitive. For example, 6-thioguanine was used to select the murine P815* mastocytoma cell line described in Example 1, and 5-bromo-2′-deoxyuridine was used to select the human 143B osteoasarcoma cell line described in Example 7. Both cell lines die when cultured in HAT-containing medium, as described in Examples 3 and 9.

As an alternative, a pre-established immortal human tumor cell line can be used, provided that at least one of the tumor-associated antigens from the patient's tumor cells are matched to these pre-established immortal tumor cells.

A sample is provided with a source of DLCs or DCs. Such samples containing these cells or their precursors include for example peripheral blood, cord blood, bone marrow, lymph or accessible lymph nodes; they may be taken from the patient or from a healthy, HLA-compatible donor. From there, two alternatives are available. Functionally-competent DLCs or DCs can be purified directly from these samples, using various methods described in the literature. Alternatively, functionally-competent DLCs or DCs can be purified after in vitro differentiation of the precursors contained in these samples, which can be done by culturing the latter in the presence of cytokines, as described hereunder.

The DLCs or DCs are prepared for cell fusion, in one of the 4 following ways:

(1°) Primary DLCs or DCs purified directly from blood, lymph or other tissues are maintained in culture for no longer than 24 hours, as described for mouse spleen DLCs in Example 2.

(2°) Primary cultured DLCs or DCs differentiated from blood, bone marrow or other tissues are cultured for at least 7 days in the presence of cytokines, as described for human blood DLCs in Example 8 or as published by Sallusto and Lanzavecchia (J. Exp. Med. 179, pp. 1109-111 (1994)); Romani et al. (J. Exp. Med. 180, pp. 83-93 (1994)); Mackensen et al. (Blood 86, pp. 2699-2707 (1995)).

(3°) Immortal DLCs or DCs can be derived from primary-cultured DLCs or DCs, for example by adapting the method described by Paglia et al. (J. Exp. Med. 178, pp. 1893-1901 (1993)). These authors immortalized neonatal mouse spleen DLCs or DCs by using a recombinant retrovirus.

(4°) HAT-sensitive variants of these DLC or DC lines can thereafter be derived by standard culture techniques, to yield drug-sensitive immortal DLCs or DCs.

A tumor cell partner is then fused with a DLC partner. From there, two alternatives are available, namely to separate or not to separate the fused cells by metabolic selection. After fusion, the treated cells include a plurality of DLC/tumor cell hybrids, as well as unfused tumor cells and unfused DLCs. If no selection is applied, fused cells as well as unfused cells are used for inducing an anti-tumor immunity in vivo and/or in vitro. If a metabolic selection is applied, for example by plating the treated cells in HAT-medium, only the immortal, HAT-resistant hybrid cells survive (Examples 3 and 9) and permanent cell lines hereafter termed DLC or DC/tumor cell hybridomas are developed from them.

The DLC or DC /tumor cell hybridomas with therapeutic potential are then selected from all growing hybridomas. Their therapeutic potential is linked to the retention of pertinent DLC or DC characteristics and of pertinent tumor cell characteristics. Pertinent DLC or DC characteristics include DLC or DC morphology, DLC or DC surface markers, DLC or DC genetic markers and the capacity to activate immune cells in vitro. At least one of these DLC or DC characteristics may suffice to qualify hybridomas made of (drug-sensitive) immortal tumor cells and primary cultured DLCs or DCs, since these hybridomas necessarily inherited immortality from the tumor parent.

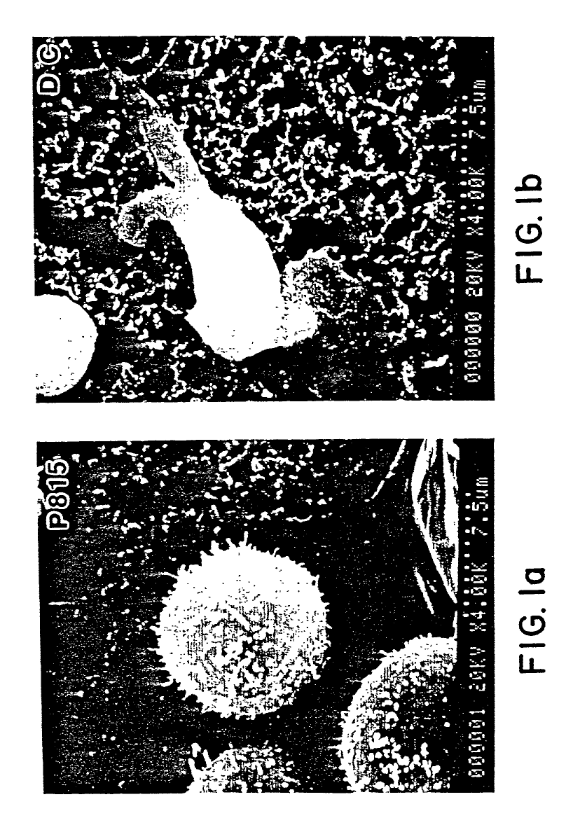

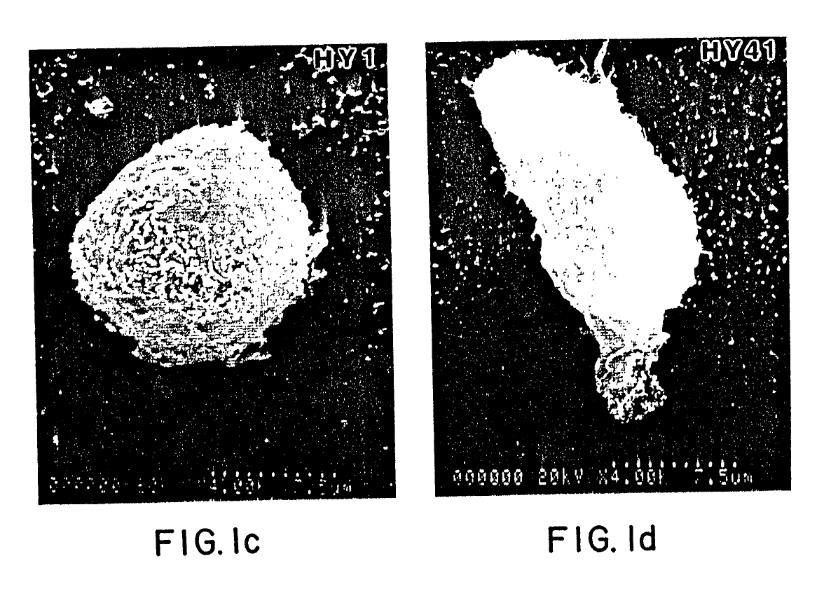

(1°) The selection may be based on the morphologic DLC or DC appearance of the hybridoma by scanning electron microscopy (SEM), as shown in Example 4A and FIG. 1. Such an analysis can be performed on a minute sample of cells at a very early stage of hybridoma development, allowing the culture efforts to be focused on the dendritic-like or dendritic hybridomas.

(2°) In the absence of morphological DLC or DC characteristics, as in Example 10A, the expression of DLC or DC surface markers may be used to select hybridomas with therapeutic potential. If such DLC or DC surface markers, including namely T-cell activating molecules, are not expressed on resting hybridomas, they may nevertheless be induced by treatment with cytokines or other activating agents, as described in Example 10B.

(3°) Genetic DLC or DC markers are further used to confirm or to exclude the contribution of a T-cell, B-cell or other cell type to the hybridoma, as in Examples 4C and 10C. HLA-DR gene typing can also be used to identify blood donor genes when the tumor cell and the DLC are from distinct individuals, as in Example 10C.

In DLC or DC/tumor cell hybridomas involving patient's related pre-established immortal tumor cells, it is necessary to select dendritic-like hybridomas that express in addition at least one of the patient's matched tumor-associated antigens. Standard immunocytochemistry can be performed on small samples of the hybridomas to identify such tumor-associated antigens as Her2/neu for breast cancer and carcinoembryonic antigen (CEA) for colon cancer. The hybridomas identified as potentially useful are amplified in culture for complete phenotypic characterization (chromosomes, genetic markers, cell surface markers and sub-cellular morphology) and for clinical use.

The various embodiments of the invention are briefly described as follows:

### Embodiments A, B, C

Primary cultured patient's tumor cells are fused with primary cultured DLCs or DCs purified from blood, lymph or other tissue (A), or with primary cultured DLCs or DCs differentiated from precursors derived from blood, bone marrow or other tissue (B), or with immortal DLCs or DCs (C), to yield a plurality of DLC or DC/tumor cell hybrids that are used without selection.

### Embodiments D, E

Primary cultured patient's tumor cells are fused with immortal DLCs or DCs (embodiment D) or with drug-sensitive immortal DLCs or DCs (embodiment E) to yield a plurality of DLC/tumor cell hybridomas; the latter are mixed in embodiment D with unfused immortal DLC or DC. In these embodiments, hybridomas with both DLC or DC characteristics and tumor cell characteristics may be selected for further use.

### Embodiments F, G

Patient's immortal tumor cells are fused with primary cultured DLCs or DCs purified from blood, lymph or other tissue (F), or with primary cultured DLCs or DCs differentiated from precursors (G), to yield a plurality of DLC or DC/tumor cell hybridomas, mixed with unfused immortal tumor cells. In these embodiments, hybridomas with DLC or DC characteristics are selected for further use.

### Embodiments H, I

Patient's drug-sensitive immortal tumor cells are fused with primary cultured DLCs or DCs purified from blood, lymph or other tissue (H), or with primary cultured DLCs or DCs differentiated from precursors (I), to yield a plurality of DLC or DC/tumor cell hybridomas. In these embodiments, hybridomas with DLC or DC characteristics are selected for further use.

### Embodiments J,K

Patient's related, pre-established immortal tumor cells are fused with primary cultured DLCs or DCs purified from blood, lymph or other tissue (J), or with primary cultured DLCs or DCs differentiated from precursors (K), to yield a plurality of DLC or DC/tumor cell hybridomas, mixed with unfused immortal tumor cells. In these embodiments, hybridomas with DLC or DC characteristics and expressing in addition the patient's matched tumor-associated antigen(s) may be selected for further use.

### Embodiments L, M

Patient's related, pre-established, drug-sensitive immortal tumor cells are fused with primary cultured DLCs or DCs purified from blood, lymph or other tissue (L), or with primary cultured DLCs or DCs differentiated from precursors (M), to yield a plurality of DLC or DC/tumor cell hybridomas. In these embodiments, hybridomas with DLC or DC characteristics and expressing in addition the patient's matched tumor-associated antigen(s) may be selected for further use.

The selected hybridomas are then used for inducing an anti-tumor immunity, either in vivo or in vitro, thereby contributing to the rejection of the residual tumor in the patient. For the induction of an anti-tumor immune response in vivo, the DLC or DC/tumor cell hybridomas are irradiated or otherwise inactivated, and injected, for example sub-cutaneously, into the patient. The patient is monitored for signs of an anti-tumor immune response and for the clinical evolution of his/her cancer. In a murine model, a single injection of a living DLC/tumor cell hybridoma into syngeneic mice elicited an anti-tumor immune response as shown in Examples 5A and 5B. In addition, multiple injections of an irradiated DLC or DC/tumor cell hybridoma had a therapeutic effect on mice preinoculated with a lethal dose of tumor cells, as shown in Example 5C. For the induction of an anti-tumor immune response in vitro, the DLC or DC/tumor cell hybridomas are irradiated or otherwise inactivated, and cultured with the immune cells of the patient. The activated immune cells are then re-injected into the patient. The patient is monitored for the presence of an anti-tumor immune response and for the clinical evolution of his/her cancer.

### EXAMPLES

The following experimental examples are provided to illustrate the invention.

### Example 1

### Preparation of Murine Tumor-Derived Cells

The P815-X2 cell line was derived from the methylcholanthrene-induced mastocytoma P815 of mouse DBA/2 origin (Dunn and Potter, 1957, J. Natl. Cancer Inst. 18: 587-601. This cell line was obtained by Thierry Boon, director of the Ludwig Institute for Cancer Research, Brussels Branch, Belgium, and recloned by his group (Uyttenhove et al, 1980, J. Exp. Med 1562: 1175-1183). The subclone P1 was extensively used by T. Boon's group and given to the present inventors in 1980. A 6-thioguanine-resistant mutant was derived from P1, as described by Le et al, 1982, Proc. Natl. Acad. Sci. USA 79:7857-7861. Briefly, P1 cells were cultured in Dulbecco's modified Eagle's medium (Grand Island Biological Co., Grand Island, N.Y.) supplemented with 10% fetal calf serum (FCS) (Gibco BRL, Merelbeke, Belgium), in a 7% CO₂ atmosphere. Increasing concentrations of 6-thioguanine (Sigma, Bornem, Belgium), ranging from 1 μg/ml to 30 μg/ml were added to the culture. The final 6-thioguanine-resistant cells died in HAT-medium, i.e. in medium supplemented with 10⁻⁴ M hypoxanthine, 3.8× 10⁻⁷ M aminopterin, and 1.6×10⁻⁵ M 2-deoxythymidine (HAT supplement, Gibco BRL) . Several HAT-sensitive clones were isolated by limiting dilution from these 6-thioguanine-resistant cells. A HAT-sensitive clone expressing MHC class I antigens was used in the present invention and will hereafter be called P815*.

P815* cells were cultured at 37° C. in a 7% CO₂ atmosphere in tissue culture flasks (Becton Dickinson, Calif.) containing RPMI 1640 medium (Seromed Biochem KG, Berlin, Germany) with 10% FCS (Gibco BRL). One day before use, P815* cells were diluted with fresh medium in order to be in exponential growth phase at the time of cell fusion.

### Example 2

### Preparation of Murine Dendritic-Like Cells from the Spleen

The preparation of splenic DLCs was done according to a multi-step procedure initially described by Crowley et al, 1989, Cell. Immunol. 118: 108-125. This procedure was adapted as described by Sornasse et al, 1992, J. Exp. Med 175:15-21. The procedure was started one day before the fusion experiment and yielded 200,000 to 500,000 DLCs per spleen.

Briefly, DBA/2 mice were obtained from Charles River, Sulzfeld, Germany, and maintained in specific pathogen-free conditions. Animals 8 to 10 weeks old were killed by cervical dislocation; their spleens were quickly removed and kept in cold RPMI 1640 medium. The spleens were digested with collagenase (CLSIII; Worthington Biochemical Corp., Freehold, N.J.) and separated into low and high density fractions on a bovine serum albumin gradient (Bovuminar Cohn fraction V powder; Armour Pharmaceutical Co., Tarrytown, N.J.). Low-density cells were cultured during 2 hours in RPMI 1640 medium with 10% FCS, and the non-adherent cells were removed by vigorous pipetting. The latter were further cultured for 1 hour in serum-free RPMI 1640 medium. The non-adherent cells were removed by gentle pipetting and cultured overnight in RPMI 1640 medium with 10% FCS. The final non-adherent fraction contained at least 95% dendritic cells, as assessed by morphology and specific staining.

### Example 3

### Fusion of Murine Tumor Cells and Dendritic-Like Cells

The procedure used to fuse HAT-sensitive tumor cells with mortal splenic DLCs was adapted from procedures used in our laboratory to generate monoclonal antibodies, as described by Franssen et al, Protides of the Biological Fluids, editor H. Peeters, Pergamon Press, Oxford, 1982, pp 645-648.

Briefly, splenic DLCs and P815* cells were extensively washed in serum-free RPMI 1640 medium. Five million DLCs were mixed with the same number of HAT-sensitive P815* cells in a 15 ml conical tube and centrifuged. Two hundred μl of a 50% solution of polyethylene glycol (PEG 4000, Merck AG, Darmstadt, Germany) in RPMI 1640 medium were added dropwise to the cell pellet. The fusion was then stopped by the stepwise addition of RPMI 1640 medium.

The cells were washed to remove the PEG and resuspended in RPMI 1640 medium with 10% FCS. After 2 hours incubation at 37° C., the cells were centrifuged, resuspended in RPMI 1640 medium containing HAT and 10% FCS, and plated at 10⁴ cells/well in flat-bottomed 96-well plates (Becton Dickinson, Calif.). The plates were seeded one day before use with a feeder layer consisting of 5,000 irradiated peritoneal cells/well. Peritoneal cells were taken from Balb/c mice and irradiated at 2,000 rads from a Cobalt 60 source before plating. The plated fusion was cultured at 37° C. in a 7% CO₂ atmosphere. The medium (RPMI 1640 with 10% FCS and HAT) was renewed as required by cell growth. In these conditions, unfused DLCs, that are not immortal, died within a few days of culture; unfused P815* cells, that are immortal but HAT-sensitive, died in the HAT-containing-medium, and only hybrid cells, combining the immortality of P815* cells with the HAT-resistance of DLCs survived and developed into growing DLC hybridomas.

After 3-4 weeks of culture, wells that contained a growing DLC hybridoma could be clearly identified by phase contrast microscopy. The content of a positive well was transferred into a larger well (24-well plates, Becton Dickinson, Calif.) previously seeded with irradiated peritoneal cells. Eventually, DLC hybridomas were transferred to small tissue culture flasks (Becton Dickinson, Calif.) and amplified for characterization and storage in liquid nitrogen.

### Example 4

### Selection of Murine Dendritic-Like Cell/Tumor Cell Hybridomas with Therapeutic Potential

The goal of these experiments was to select DLC/tumor cell hybridomas exhibiting at least one of the three following characteristics:

(1°) a DLC morphology;

(2°) DLC surface markers;

(3°) DLC genetic markers.

### A. Dendritic-Like-Cell Morphology

P815* tumor cells, fresh splenic DLCs, and DLC/tumor cell hybridomas were analyzed by scanning electron microscopy (SEM). About one million cells were fixed in 2-4% glutaraldehyde for 24 hours at room temperature and washed in phosphate buffer saline. Cell suspensions were then collected on 0.2 μM nylon filters, postfixed in 1% osmium tetroxide followed by 1% tannic acid mordant and uranyl acetate, with a series of saline washes in between each step. The samples were dehydrated through graded alcohols, then critical point dried from CO₂. After critical point drying, the samples were mounted on aluminium stubs and sputter coated with gold using a Bio-Rad PS3 coating unit. The cells were examined at 20 kV in a Hitachi S520 scanning electron microscope.

Photographs of the cells are shown in FIG. 1. In these conditions, P815* tumor cells appeared as uniform rounded cells, whose surface was spiked with numerous short microvilli (FIG. 1a). In contrast, splenic DLCs appeared as irregular cells, due to the presence of clearly-visible cytoplasmic extensions, resembling pseudopodia and veils. Furthermore, the DLC surface was not spiked with numerous microvilli, but displayed instead fewer, larger protrusions. The hybridoma cells were in general much larger than the parent P815* cells. Many of them (like the one named HY1) looked very much like the P815* parent, which was linked to their round regular shape and microvilli-like protrusions (FIG. 1c). In contrast, hybridomas HY41 and HY62 looked much more like the DLC parent, when considering their irregular shape and relatively bare cell surface with some large protrusions, as shown for HY41 in FIG. 1d. However, a DLC morphology may be assumed not only by dendritic cells of myeloid origin, but also by cells derived from other lineages, including cells of the B- and T- lymphocyte lineages, like follicular dendritic cells and dendritic epidermal T-cells, respectively. In order to determine the cell lineage of the DLC that fused with the P815* tumor cell, other DLC characteristics were investigated for hybridomas HY41 and HY62.

### B. Dendritic-Like-Cell Surface Markers

Cell surface molecules were characterized by FACS analysis, as described by Flamand et al, 1990, J. Immunol 144:2875-2882. Briefly, the cells were preincubated with 2.4G2, a rat anti-mouse Fc-receptor (Fc-R) monoclonal antibody (mAb) for 10 min prior to staining with fluorescein-coupled monoclonoal antibody (fl. mAb). This preincubation was done to prevent the non-specific binding of mAb to cellular Fc-R. When unlabelled mAb were used, they were revealed by incubation with fluoresceinated anti-IgG antibodies. The labelled cells were gated for size and side scatter to eliminate dead cells and debris, and analyzed on a Facscan (Becton Dickinson, Calif.).

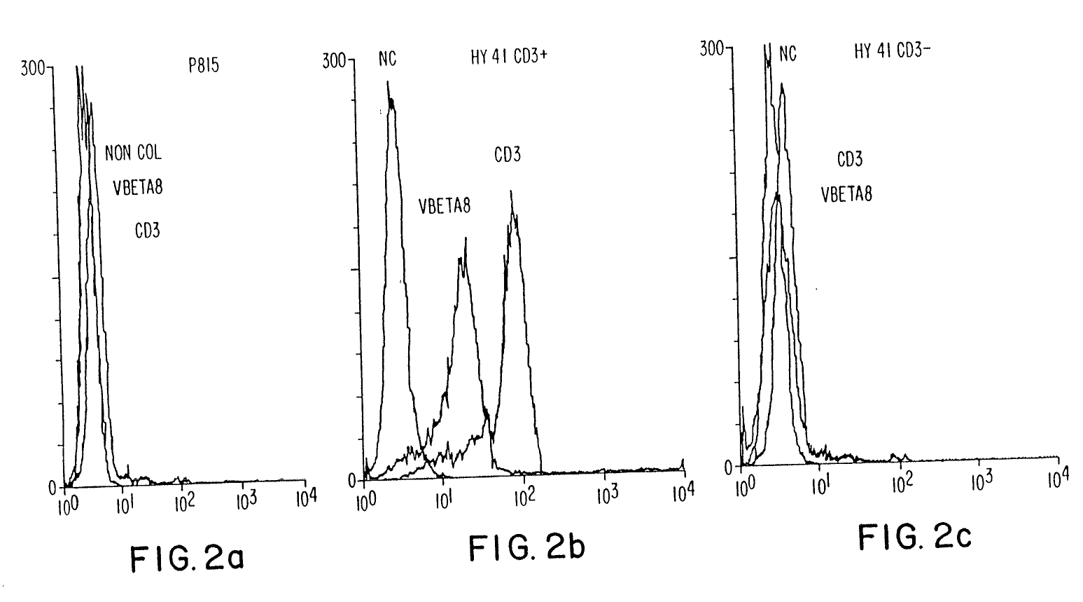

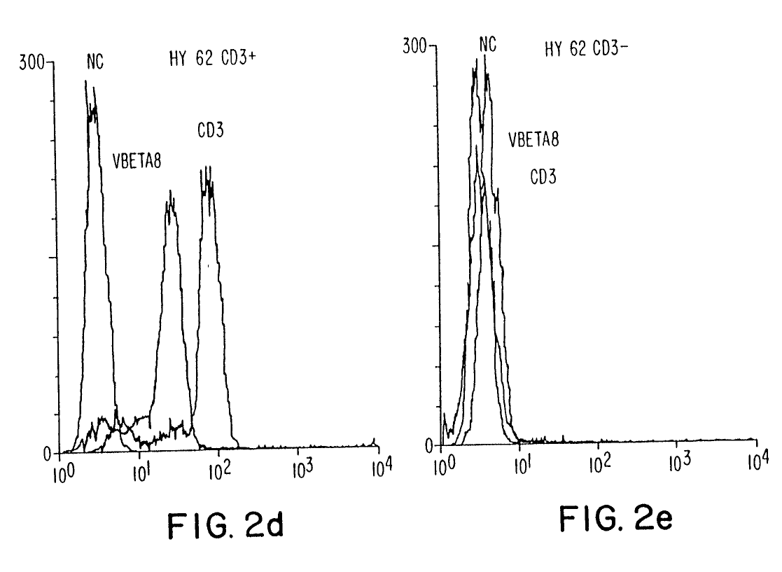

The results are summarized in Table 1. No T-cell activating molecules or other dendritic-cell-associated molecules were expressed by the HY41 and HY62 hybridomas. However, a fraction of the cells of both hybridomas expressed surface CD3e chains of the T-cell receptor (TCR), suggesting that they were T-lymphocyte/tumor cell hybridomas. After cloning by limiting dilution, CD3+ and CD3− subclones were isolated from both hybridomas. FIG. 2 shows that the HY41 and HY62 CD3e+ subclones were also labeled by a fl mAb specific for the V b8 domain of the TCR, whereas P815* tumor cells and the CD3e-subclones remained unstained. These results showed that the HY41 and HY62 hybridomas expressed an a/b TCR, and hence had incorporated a dendritic-like T-lymphocyte. However, neither CD4 or CD8 were expressed by the hybridomas. In order to confirm these cell surface marker studies, genetic marker studies were undertaken.

TABLE 1
Cell Surface Markers of Murine Dendritic-Like-
Cell/Tumor Cell Hybridomas HY41, HY62 and Parent Cells

| | | DLCs | | | |
|---|---|---|---|---|---|
| Reagents | Surface markers | ⁽¹⁾ | P815* | HY41 | HY62 |
| | Present on DLCs and | | | | |
| | P815* | | | | |
| 31.3.4 mAb | MHC class I Kd | + | + | + | + |
| 34.4.20 mAb | MHC class I Dd | + | + | + | + |
| 30.5.7 mAb | MHC class I Ld | + | + | + | + |
| 3E2 f1 mAb | ICAM-1 (CD54) | + | + | − | − |
| | Present on P815* only | | | | |
| 2.4G2 mAb | Fc-R | − | + | − | − |
| | Present on DLCs only | | | | |
| | T-cell activating | | | | |
| | molecules: | | | | |
| 14.4.4 f1.mAb | MHC class II | + | − | − | − |
| 16-10A1 f1 mAb | B7-1 (CD80) | + | − | − | − |
| GL1 f1 mAb | B7-2 (CD86) | + | − | − | − |
| CTLA4- human Ig | CTLA4-ligand | + | − | − | − |
| M1/69 f1 mAb | HSA (CD24) | + | − | − | − |
| | Other molecules: | | | | |
| N418 f1 mAb | N418 (CD11c) | + | − | − | − |
| 145-2 C11 | CD3ε | nd⁽²⁾ | − | + | + |
| F23-1 | TCR V β8 chain | nd | − | + | + |
| H129.19 | CD4 | nd | − | − | − |
| 53-6.7 | CD8a | nd | − | − | − |

⁽¹⁾By cell scatter and cell surface marker analyses, DLCs contained more than 95% dendritic cells;
⁽²⁾nd: not detectable

31.3.4, 34.4.20, 30.5.7: mouse anti-mouse H2-K^{d}, D^{d} and L^{d} mAb, respectively; Ozato et al, 1980, J. Immunol. 124:533-; 3E2: hamster anti-ICAM-1, from Pharmingen, San Diego, Calif. 2.4G2 : rat anti-mouse Fc-gamma-RII/III mAb (Unkeless, 1979, J. Exp. Med. 150:580-586;

14.4.44: mouse anti-I-E^{d} fluorescein-coupled mAb (fl mAb); Ozato et al, 1980, J. Immunol. 124:533-16-10A1: rat anti-B7-1 fl mAb; Razi-Wolf et al, 1993, Proc. Natl. Acad. Sci. USA 90:11182-11186;

GL1: hamster antiB7-2 fl mAb; Hathcock et al, 1993, Science 262:905-907;

CTLA4-human IgG fusion protein: Linsey et al, 1991, J. Exp. Med. 174:561-569;

M1/69: Rat anti-HSA, from Pharmingen, San Diego, Calif. N418: hamster anti-mouse CD11c; Metlay et al, 1990, J. Exp. Med. 171:1753-1771;

145-2C11: hamster anti-mouse DC3e fl mAb; Leo et al, 1987, Proc. Natl. Acad. Sci. USA 84:1374

F23.1: mouse anti-mouse TCR V b8 fl mAb from ATCC, Bethesda Md.

H129.19: rat anti-mouse CD4 fl mAb, from Gibco BRL, Gaithersburg, Md.

53-6.7: rat anti-mouse CD8a fl mAb, from Gibco BRL, Gaithersburg, Md.

ND: not detectable

### C. DLC Genetic Markers

First, Southern blot analysis was used to analyse the rearrangement status of the TCR genes in genomic DNA from the HY41 hybridoma. The mouse T-cell hybridoma 13.26.8-H6 was used as a reference for rearranged TCR genes (Ruberti et al, 1992, J. Exp. Med. 175: l57-162), and P815* mastocytoma cells as well as DBA/2 spleen cells were taken as controls for germ line TCR genes. Genomic DNA was extracted from 2×10⁷ cultured cells and from spleens, using the Genome DNA Kit (Bio 101, CA, USA) according to the manufacturer's instructions. 10 mg of DNA were digested for ±4 hours with various restriction enzymes, separated on a 1% agarose gel and transferred to a nylon membrane (Qiabrane Nylon plus, Qiagen, Hilden, Germany) according to standard procedures. The blot was hybridized to a DIG-labeled synthetic oligonucleotide of 50 bases targeted to the first exon of the constant region of the mouse TCR b chain and processed for chemiluminescent detection using Boehringer Mannheim's DIG detection kit. The results showed that the HY41 genome contained a rearranged TCR b chain gene, which is a hallmark of T-cell lineage commitment (not shown).

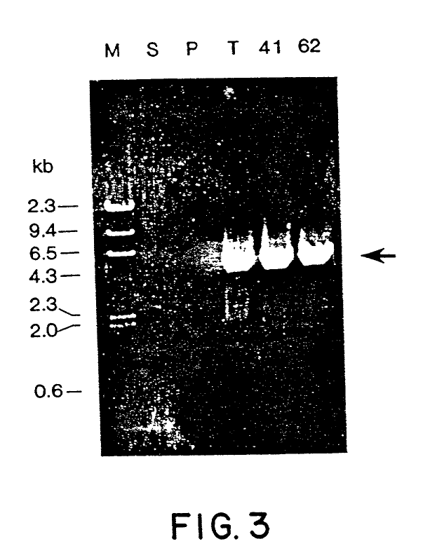

Next, the Polymerase Chain Reaction (PCR) was used to detect rearranged V b8-Cb sequences of the TCR in genomic DNA. The upstream primer was targeted to bases 47-66 with respect to the ATG initiation codon of the mouse V b8 region (5′-AACACATGGAGGCTGCAGTC-3′) and the downstream primer was targeted to bases 141-160 of the fisrt exon of the Cb region (5′-GTGGACCT CCTTGCCATTCA-3′). The PCR was carried out essentially according to the instructions of Boehringer Mannheim's Long Range Expand PCR System. Analysis of the PCR products on a 1% agarose gel stained with ethidium bromide is shown in FIG. 3. A fragment with the expected length (4.5 to 5 kb)of the rearranged Vb8-Cb fragment is clearly seen in DNA from the T-cell hybridoma 13-26-8-H6 (lane T), used as a positive control, as well as in DNA from the HY41 and HY62 hybridomas (lanes 41 and 62); this fragment is not amplified in DNA from P815* tumor cells and from spleen cells (lanes P and S), used as negative controls. These results confirm that the DLC that fused with a P815* tumor cell to yield the HY41 and HY62 hybridomas was a T-lymphocyte expressing an a/b TCR receptor, including the Vb8 domain. These hybridomas will hereafter be termed T-DLC/tumor cell hybridomas.

In conclusion, the HY41 and HY62 T-DLC/tumor cell hybridomas were selected for further studies because of their DLC morphology and T-lymphocyte lineage. In both hybridomas, the T-lymphocyte fusion partner was a rare and undetectable contaminant of the splenic DLC preparation. In view of the complex genetic regulations controling CD4 and CD8 expression in somatic cell hybrids (Wilkinson et al, 1991, J. Exp. Med. 174: 269-280), it is impossible to determine a posteriori if the fusing T-cell was a CD4⁺, CD8⁺, or CD4-CD8-“double negative” T-cell. However, whatever the sublineage of T-lymphocyte involved, the next step was to determine the in vivo immunogenicity of these T-DLC/tumor cell hybridomas.

### Example 5

### In Vivo Immunogenicity of Murine T-Dendritic-Like-Cell/Tumor Cell Hybridomas

The goal of these experiments was to determine if the hybridomas induced an efficient immune rejection in vivo, as measured by the following criteria:

(1°) rejection of the hybridomas by immunocompetent mice;

(2°) vaccination with the hybridomas against a subsequent inoculation of tumor cells;

(3°) treatment with the hybridomas after prior inoculation of tumor cells.

### A. Immune Rejection of T-DLC/Tumor Cell Hybridomas

Groups of 10 to 12 DBA/2 mice were injected intra-peritoneally with 500,000 living cells of the P815* tumor or of the HY41 hybridoma. Injected animals included mice immunosuppressed by sub-lethal irradiation as well as immunocompetent mice. All irradiated animals died from their tumor within four weeks of inoculation, showing that the HY41 and P815* cell lines were very similar in their tumorigenicity (FIG. 4). In contrast, 9/12 (75%) immunocompetent animals injected with the HY41 hybridoma survived two months after inoculation, when only 2/10 (20%) mice had survived the parental tumor injection. This experiment showed that the HY41 hybridoma was as tumorigenic as the parent tumor in irradiated mice, but more immunogenic than P815* in immunocompetent mice. Similar results were obtained with hybridoma HY62 (not shown).

### B. Induction of Tumor Resistance by Murine T-DLC/Tumor Cell Hybridomas

The 9 surviving HY41-treated mice, as well as 9 untreated animals, were challenged intra-peritoneally with 500,000 P815* cells. All (9/9) untreated mice died from their tumor within six weeks of inoculation, showing that the tumor cell injection was lethal for unimmunized animals. By contrast, 7/9 HY41-treated animals were still alive 6 weeks after tumor challenge, and 4/9 of them survived for at least three months (FIG. 5). These results strongly suggested that prior treatment of syngeneic mice with living HY41 DC hybridoma cells induced a memory immune response against the parent P815* cell line, conferring tumor resistance to 44% of the treated animals. A similar tumor-resistance could be induced by the injection of living HY62 hybridoma cells (not shown).

The spleens of the 4 P815*-resistant mice were tested in vitro for the presence of anti-P815* cytotoxic T-cells, as described by Moser et al, 1987, J. Immunol 138: 1355-1362. Briefly, spleen cell suspensions were stimulated in vitro during 5 days with the irradiated P815* cells, in order to induce a measurable memory response. They were -then used as effector cells on chromium-loaded P815* and L1210 target cells. The latter have the same MHC class I haplotype (H-2d) as P815 cells. At several effector/target ratios, the spleen cells of the untreated animals completely failed to lyse the P815* and the L1210 target cells (FIGS. 6A and 6B). In contrast, the spleen cells from the 4 P815*-resistant mice lysed efficiently and specifically the P815* targets, without showing any significant activity on the L1210 targets. These results showed that the HY41-treated, P815*-resistant animals were able to mount a strong and tumor-specific cytolytic response upon in vitro restimulation.

### C. Induction of Tumor Treatment by Murine T-DLC/Tumor Cell Hybridomas

In this experiment, 40 DBA/2 mice received an ip injection of 2×10⁵ P815* tumor cells. Seven days later, the mice were divided into 4 groups of 10 animals; the first group was left untreated while the 3 other groups were treated by 4 weekly ip injections of 2×10⁶ irradiated (15,000 F) P815*, HY41 or HY62 cells. The data are presented in FIG. 7. Untreated animals all died within 7 weeks of tumor inoculation, and 20% of the mice treated with irradiated P815* tumor cells survived, confirming the weak immunogenicity of the P815 tumor cell line. In contrast, 60% and 40% of the mice treated with irradiated HY41 and HY62 hybridoma cells, respectively, survived the prior injection of a lethal dose of P815* cells. These data showed that hybridoma HY41, and to a lesser extent hybridoma HY62, could induce the immune rejection of an established tumor. However, the mechanism leading to such an efficient in vivo immune rejection remained unclear. One possibility that was explored concerned the secretion of immunomodulating cytokines.

### Example 6

### In Vitro Analysis of Cytokine Expression by T-DLC/tumor Cell Hybridomas

The goal of these experiments was to determine whether the HY41 and HY62 hybridomas synthesized some cytokines that could account, at least in part, for their in vivo immunogenicity. Total RNA was prepared from activated spleen cells, from P815* tumor cells and from the HY41 and HY62 hybridomas according to standard procedures. The Reverse-Transcription Polymerase Chain Reaction (RT-PCR) and cytokine-specific primers were used to amplify IL-2, IL-4, IL-10 and interferon γ (IFN-γ) mRNA sequences, as described by De Wit et al, J. Immunology, 1993, 150: 361-366. The primers used to amplify IL-12 p40 sequences were 5′-TTCAACATCAAGAGCAG TAGC-3′ and 5′-GGAGAAGTAGGAATGGGGAGT-3′. Analysis of the RT-PCR products on ethidium bromide-stained agarose gels showed that P815* tumor cells constitutively expressed IL-4 mRNA and that the HY41 and HY62 hybridomas constitutively expressed IL-2 and IL-4 mRNAs, but not IL-10, IL-12, and IFNg mRNAs. These cytokine mRNAs were nevertheless detected in activated spleen cells, used as a positive control. In conclusion, these data showed that the HY41 and HY62 T-DLC/tumor cell hybridomas constitutively expressed IL-4 like the parent P815* tumor cell, and IL-2, like the parent T-lymphocyte. These cytokines, if secreted in vivo, may at least partially contribute to the immunogenicity of the hybridomas.

### Example 7

### Preparation of Human Tumor-Derived Cells

The human 143B thymidine kinase negative osteosarcoma cell line (hereafter termed 143B) is a HAT-sensitive cell line that was purchased from the ATCC (CRL No. 8303). The cells were cultured in Dulbecco's modified Eagle's medium supplemented with 10 FCS, 2% penicillin/streptomycin, 1% sodium pyruvate (all from Gibco BRL, Merelbeke, Belgium) and 0.015 mg/ml of 5-bromo-2′-deoxyuridine (Sigma Chemical Co, St Louis, Mo.). One day before fusion, the cells were diluted with fresh medium in order to be in exponential growth phase.

### Example 8

### Preparation of Human Dendritic-Like Cells from Peripheral Blood

Dendritic cells were differentiated in vitro from adherent blood precursors, using an adaptation of the technique described by Romani et al, 1994, J. Exp. Med. 180:83-93. Briefly, peripheral blood mononuclear cells (PBMC) were isolated from the buffy coat of a healthy donor by density gradient centrifugation on lymphoprep (Gibco BRL). Adherent cells were prepared by plating 10⁷ PBMC on 6-well tissue culture plates in 3 ml RPMI supplemented with 200 mM L-Glutamine, 50 mM Mercaptoethanol and 10% FCS. After 2 hours incubation at 37° C., the non-adherent cells were discarded by a very gentle rinse, and the adherent cells were further cultured in the above-described medium supplemented with GM-CSF (Leucomax, 800 U/ml) and IL-4 (Genzyme, 500 U/ml), at 37° C. in a humidified atmosphere with 5% CO₂. After 7 days of culture, DLCs were recovered and characterized by cell scatter and cell surface marker analysis. The DLCs used for fusion contained 50% of monocytic-like cells, expressing CD14 but not CD1a or CD1c, as well as 38% of T-lymphocytes, 4% of NK cells and 8% of B lymphocytes.

### Example 9

### Fusion of Human Tumor Cells and Dendritic-Like Cells

The procedure used to fuse HAT-sensitive tumor cells with DLCs was adapted from procedures used to generate monoclonal antibodies (Current Protocols in Immunology, chapter 2.5.4). The 143B tumor cells and the DLCs were extensively washed in serum-free medium (RPMI 1640); 2×10⁶ DLCs were mixed with 1×10⁶ 143B osteosarcoma cells and centrifuged. The pellet was resuspended in 500 μl of a 50% solution of polyethylene glycol (PEG 4000, Gibco) in Dulbecco's phosphate buffered saline without Ca⁺⁺, Mg⁺⁺ (ref. 14030035). After 1 minute, the PEG was progressively diluted by the slow and progressive addition of serum-free medium. The cells were washed free of PEG and resuspended in RPMI 1640 with 10% FCS. They were eventually plated at 2×10⁴ cells/well in flat-bottomed 96-well plates (Falcon, Becton Dickinson) and cultured in a 5% CO₂ atmosphere at 37° C. HAT medium was added to the wells 24 hours after fusion and renewed every two days. In these conditions, unfused DLCs died within 2-3 weeks of culture, unfused 143B osteosarcoma cells died in HAT-medium and only hybrid cells combining the immortality of the tumor cell with the HAT-resistance of a DLC survived and developed into growing cell lines. After 3-4 weeks of culture, wells containing growing cell lines were clearly identified by phase contrast microscopy. Their contents were transferred into larger wells and eventually into culture flasks for amplification. Culture stocks were frozen in liquid nitrogen before analysis.

### Example 10

### Identification of Human Dendritic-Like Cells/Tumor Cell Hybridomas with Therapeutic Potential

The goal of these experiments was to identify human DLC/tumor cell hybridomas presenting at least one of the three following characteristics:

(A) DLC morphology;

(B) DLC surface markers;

(C) DLC genetic markers.

### A. DLC Morphology

The 143B osteosarcoma cells and a series of hybridoma cells were analyzed by SEM, as described in example 4. Comparison of the parent cells and hybridoma cells showed that none of the hybridomas analysed, including F3BG10 cells, displayed morphologic dendritic-like features. In the absence of such features, other dendritic-like features were analyzed, namely the presence of DLC surface markers.

### B. DLC Surface Markers

Cell surface markers were analyzed as described in Example 4. Results are summarized in Table 2. None of the tested hybridomas, including F3BG10 cells, expressed the T-cell activating molecules HLA-DR, B7.1, and B7.2. However, they expressed HLA class I, ICAM-1 (CD54) and LFA-3 (CD58), which were also present on the 143B tumor cells. They failed to express typical dendritic-cell markers like CD1a and CD1c, as well as markers specific for T-cells (CD3), B-cells (CD19), NK cells (CDS6) and monocytes (CD14).

Since the hybridomas tested failed to express constitutively T-cell activating molecules, they were stimulated with a variety of cytokines in order to induce such expression. It was found that 40% of the F3BG10 hybridoma cells were induced to express varying amounts of surface HLA-DR after a 24 hour incubation with interferon γ. After cloning by limiting dilution, subclones were tested for their capacity to express induced HLA-DR. FIG. 8 shows that at least 90% of H12 cells clearly expressed induced HLA-DR, which greatly increases their immunogenic potential.

TABLE 2
Cell Surface Markers of Human Dendritic-Like-
Cell/Tumor Cell Hybridoma F3BG10 and Parent Cells

| | | DLCs | | |
|---|---|---|---|---|
| Reagents from | Surface markers | ⁽¹⁾ | 143B | F3BG10 |
| | Present on DLCs and | | | |
| | 143B | | | |
| Pharmingen | HLA class I | + | + | + |
| Immunotech | ICAM-1 (CD54) | + | + | + |
| Becton | LFA-3 (CD58) | + | + | + |
| Dickinson | | | | |
| | Present on DLCs only | | | |
| | T-cell activating | | | |
| | molecules: | | | |
| Becton | HLA-DR | + | − | −⁽²⁾ |
| Dickinson | | | | |
| Innogenetics | B7.1 (CD80) | + | − | − |
| Pharmingen | B7.2 (CD86) | + | − | − |
| | Other molecules: | | | |
| Immunotech | CD1a | + | − | − |
| Immunotech | CD1c | + | − | − |
| Becton | CD14 | + | − | − |
| Dickinson | | | | |
| Becton | CD2 | + | − | − |
| Dickinson | | | | |
| Becton | CD3 | + | − | − |
| Dickinson | | | | |
| Becton | CD19 | + | − | − |
| Dickinson | | | | |

⁽¹⁾: By cell scatter and cell surface marker analyses, DLCs contained 50% monocytes, 38.% T-lymphocytes and 4% NK cells; the suspension also contained 8% of B-lymphocytes.
⁽²⁾: HLA-DR expression could be induced in 40% of F3BG10 cells and in 90% of its H12 subclone by incubation with interferon γ.

### C. DLC Genetic Markers

The goal of this first experiment was to determine whether the F3BG10 hybridoma had been generated by the fusion of a DLC with the 143B tumor cell, and to exclude that it was a revertant 143B tumor cell clone, that had become resistant to HAT-medium by mutation. This was done by typing the HLA-DR genes of the blood donor, of the 143B tumor cell and of the F3BG10 hybridoma. Genomic DNA was prepared according to standard procedures from 143B tumor cells, from the PBMC of the blood donor and from F3BG10 hybridoma cells. These DNAs were submitted to a non-isotypic HLA-DR B oligotyping method, described for the typing of DR B 1, 3, 4, 5 alleles by Buyse et al. 1993, Tissue Antigens 41: 1-4. The polymorphic second exon of the corresponding genes was amplified by PCR, and biotinylated nucleotides were incorporated into the amplifying fragments during this procedure. -The PCR products were hybridized with a combination of 31 sequence-specific oligonucleotide probes, immobilized in parallel lines on membrane strips. After a stringent wash, streptavidin-labelled alkaline phosphatase was added to mark the biotinylated DNA fragments. The addition of the BCIP/NBT chromogen resulted in a colored precipitate. All reagents were part of the Innolipa DRB Key kit purchased from Innogenetics (Zwijndrecht, Belgium). The F3BG10 lane showed a mixture of bands corresponding to alleles present in the 143B osteosarcoma cells and in the PBMC of the blood donor, confirming that F3BG10 hybridoma was a DLC/tumor cell hybridoma.

The goal of the second experiment was to investigate whether it was a T-lymphocyte or a B-lymphocyte that fused with a 143B tumor cell to yield the F3BG10 hybridoma. Genomic DNA was tested for the presence of rearranged T-Cell Receptor (TCR) genes or B-cell Receptor (BCR) genes by Southern blot analysis with TCR-specific or BCR-specific probes. Standard procedures were used. Briefly, samples of 10 mg of DNA were submitted to overnight digestion at 37° C. with different restricition enzymes. Hind3, Xbal and Hind3+Xbal were used for the TCR rearrangements and EcoRl, Hind3 and Hind3+BamHl were used for BCR rearrangements. The restriction fragments were separated by electrophoresis on a 1% agarose gel, transferred to nitrocellulose, baked and hybridized with probes specific for either the b chain gene of the TCR, or for a segment of the J gene of the Ig heavy chain of B lymphocytes. The results clearly showed that there were only germ line TCR genes and germ line BCR genes in the genomic DNA of the F3BG10 hybridoma. These data excluded that the DLC/tumor cell hybridoma F3BG10 was produced by the fusion of the tumor cell with a T-lymphocyte or a B-lymphocyte. The DLC fusion partner could have been a monocyte, a dendritic cell, an intermediate cell between these two cells, a natural killer cell or another unidentified non-B cell. Because the pattern of cytokine secretion could provide indications on the cell lineage of the fusion partner, we investigated cytokine secretion by F3BG10 cells.

### Example 11

### In Vitro Analysis of Cytokine Secretion by Human DLC/tumor Cell Hybridoma

The culture supernatants of the F3BG10 hybridoma cells and of the 143B tumor cells were assayed by ELISA for the presence of various cytokines, before and after 36 hours of culture in the presence of various stimuli including interferon γ, TNFα, GM-CSF and combinations of these. The results showed that the 143B osteosarcoma cells and the F3BG10 hybridoma cells secreted similar levels of IL-6 and IL-8, that could be increased for both cytokines by stimulation with the above-mentioned cytokines. In addition, the F3BG10 cells but not the tumor cells secreted significant levels of GM-CSF, that could be increased by stimulation. Neither the tumor cells or the hybridoma cells secreted detectable levels of IL-1β, IL-10, IL-12 and TNFα. These results showed that the F3BG10 hybridoma secreted IL-6 and IL-8 like the parent tumor cell, and GM-CSF like the parent DLC. Since it was excluded that the latter was a T-lymphocyte, this result suggested that the fusion partner was a monocyte.

### Example 12

Female DBA/2 (H-2^{d}) and CBA/J (H-2^{k}), 6-8 week old, were purchased from Charles River Wiga (Sulzfeld, Germany) and maintained in our own pathogen-free facility.

The tumor cell line is the methylcholanthrene-induced mastocytoma P815 of DBA/2 origin, derived from a 6-thioguanine-resistant mutant, according to a procedure described by Lethé et al. (1992). Briefly, P815 cells were cultured in DMEM supplemented with 10% FCS and increasing concentrations of 6-thioguanine (Sigma, St. Louis, Mo.), ranging from 1 to 30 μg/ml. The final 6-thioguanine-resistant cells died in HAT-medium, i.e. in medium supplemented with 10⁻⁴ M hypoxanthine (Merck, AG, Darmstadt, FRG), 3,8 10⁻⁷ M aminopterin (ICN Nutritional Chemicals) and 1.6×10⁻⁵ M 2-deoxythymidine (Merck AG). L1210 is a lymphocytic leukemia which arose in a DBA/2 female following painting the skin with methylcholanthrene (available through ATCC). The I-E^{d} restricted, pork-insulin specific T cell hybridoma B8P4.1C3 (24) was obtained from Dr Delovitch (J. P. Robarts Research Institute, Ontario, Canada).

Dendritic cells were generated from bone marrow progenitors according to a procedure modified from a protocol of Inaba et al. (1992) and Zorina et al. (1994). Briefly, bone marrow was flushed from tibias and femurs and depleted of lymphocytes, granulocytes and class II positive cells using a cocktail of mAbs and sheep anti-rat IgG DYNABEADS M-450 (Dynal, Oslo, Norway). The mAbs were anti-CD8, anti-CD4, GR-1 anti-granulocyte, anti-B220/CD45R, anti-I-A^{d}/I-E^{d} (Pharmingen, San Diego, Calif., USA). Cells were plated in 24-well culture plates (2.5×10⁵ cells/ml/well) in DMEM supplemented with 10% heat inactivated FCS, additives, 200 ng/ml GM-CSF and 100 U/ml TNFα, and cultured for 10 days. The cultures were fed every other day by gently swirling the plates, removing 75% of medium and adding fresh medium containing GM-CSF and TNFα. Non-adherent cells were collected at 10 days and comprised mainly dendritic cells, as assessed by morphology and specific staining using N418 (26), anti-class II, anti-B7-l (9) and anti-B7-2 (10) mAbs.

2×10⁶ DC were mixed with 2×10⁶ HAT-sensitive P815 cells in a 15 ml conical tube. The cells were washed in RPMI 1640 and pelleted by centrifugation. The fusion was started by adding dropwise, in 90 seconds, 200 μl of a 50% solution of PEG 4000 (Merck) in RPMI 1640 medium. The fusion was stopped by the stepwise addition of RPMI medium. The cells were centrifuged, resuspended in medium containing 10% FCS and additives, and incubated for 2 h, at 37° C. in 7% CO₂. The cells were centrifuged, resuspended in selection medium (RPMI 1640 containing HAT, 10% FCS and additives), and plated at 10⁴ cells/well in flat-bottomed 96-well plates (Becton Dickinson, Calif., USA). The plates were seeded 1 day before use with a feeder layer consisting of 5,000 (irradiated peritoneal cells/well. The plated fusion was cultured at 37° C. in a 7% CO₂ atmosphere. The medium was renewed as required by cell growth.

The use of lethally irradiated tumor cells as a therapeutic modality should be transferred readily into clinical application. High numbers of dendritic cells can be derived from progenitors in humans (Caux et al. (1992)). The great majority of tumor antigens are either unknown or indeterminate with regard to their immunogenic T-cell epitopes. Furthermore, the method and composition of the invention combine several advantages such as the presence of costimulatory molecules, the ability to present antigen through the exogenous (MHC class II) and endogenous (MHC class I) pathways independently from known MHC/epitope associations. Of note, presentation of multiple antigen derived epitopes may enhance anti-tumor immunity and minimize the emergence of resistant variants. Using DC as an adjuvant for antigen delivery has potential advantages over other forms of immunization in that DC may have the unique property to migrate to areas rich in T-lymphocytes and to express a variety of signals that lead to optimal activation of naive and memory cells.

### Flow Cytometry

Cells were analyzed by flow cytometry with a FACScan cytometer (Becton Dickinson and CO, Mountain View, Calif.). The cells were preincubated with 2.4G2 (a rat anti-mouse Fc receptor mAb) for 10 min before staining to prevent antibody binding to FcR, and were incubated with fluoresceinated 14-4-4 (murine IgG2a anti-I-E^{d}, available through ATCC, Rockville, Md., USA), N418 (hamster anti-mouse CD11c, 26), 16A1 (hamster anti-mouse B7-1, 9), GL1 (rat IgG2a anti-mouse B7-2, 10), anti-Heat Stable Antigen (HSA, Pharmingen, San Diego, Calif., USA), anti-mouse ICAM-l/CD54 (Pharmingen). Staining with irrelevant isotype-matched antibodies was negative on all cell types.

### PCR Analysis of P1A Gene Expression

Total RNA was extracted from P815 and hybrid cells using TRIZOL reagent (total RNA isolation reagent, Gibco BRL, Merelbeke, Belgium). Less than 1 μg RNA was used to perform a control PCR for actin and a P1A gene specific PCR with the TitanTM One tube RT-PCR System (Boehringer Mannheim, Brussels, Belgium). The cDNA synthesis was performed following the manufacturer's instructions. The PCR reactions for actin: 94° C. 2′(94° C. 30″, 60° C. 30″, 72° C. 1′20″) 40 cycles, 72° C. 10′and for P1A: 94° C. 2′(94° C. 30″, 55° C. 30″, 72° C. 30″) 35 cycles, 72° C. 10′ were in a Perkin-Elmer/Cetus DNA thermal cycler. Primers used were as follows: actin sense primer 5′-TGCTATCCAGGCTGTGCTAT-3′, actin antisense primer 5′-GATGGAGTTGAAGGTAGTTT-3′, P1A sense primer 5′-GGGACCATGGCCCACAGTGGCTCAGGT-3′ and P1A antisense primer: 5′-GGGGGATCCTTAGACAGAGGACATGCGCTTG-3′, resulting in an amplified fragment of 240 bp.

### In Vitro Responses

The complete medium used in all experiments was RPMI 1640 (Seromed Biochem KG, Berlin, Germany) or DMEM (Gibco BRL, Merelbeke, Belgium) supplemented with 10% FCS, 2% ultroser HY (a serum-free medium supplement purchased from Gibco BRL) or 1% heat-inactivated mouse serum, penicillin, streptomycin, non-essential aminoacids, sodium pyruvate, 2-ME, and L-glutamine (Flow ICN Biomedicals, Bucks, UK).

Mixed lymphocytes reaction (MLR): Splenic CD4⁺ T-cells (CBA/J, H-2^{k}) were purified by depletion of adherent cells by passage over Sephadex G10 (Pharmacia Bioprocess, Uppsala, Sweden) and complement-mediated lysis with a cocktail of anti-B220 and anti-CD8 mAbs. 2×10⁵ CD4⁺ T-cells were stimulated with increasing numbers of γ-irradiated (15,000 rads) allogeneic P815 or hybrid cells, or with γ-irradiated (3000 rads) bone marrow-derived DC. Proliferation was assessed by thymidine incorporation during the last 16 h of a 4 day-culture. The supernatants were collected after 48 h of culture, frozen and assayed for IL-2 content using a standard bioassay with an IL-2 sensitive, IL-4 insensitive subclone of the CTL.L line. In some experiments, purified blocking antibodies were added at a final concentration of 5 μg/ml, as indicated in FIG. 11.

Tumor specific immune response: resistant mice (injected with live P815 and irradiated hybrid cells, and further challenged with live P815 cells harvested from ascites (see FIG. 13) were killed 3 months after the last treatment. 6×10⁶ splenocytes were stimulated with 10⁵ irradiated (15 000 rads) P815 in a volume of 2 ml of DMEM containing additives and 2% ultroser HY. After 5 days of culture, the effectors generated were tested for lytic activity in a 3.5-h ⁵¹Cr-release assay on P815. Results are expressed as percent specific lysis at various E/T ratios. Percent specific lysis of target cells was calculated as follows: 100×(experimental release−spontaneous release)/(maximum release−spontaneous release). Each point represents the mean percent specific ⁵¹Cr release from three replicate wells. Standard errors were consistently <5% of the mean values. 50 μl of supernatants were collected after 24 h of culture, frozen and assayed for IL-2 content. IL-2 production by cells from the peritoneal cavity was tested as follows : the cells were harvested from the same treated mice by extensive washing of the peritoneal cavity with cold DMEM, and 6×10⁴ peritoneal exudate cells were cultured (in DMEM containing 1% mouse serum and additives) with various numbers of irradiated P815 cells in round-bottom 96-well plates. The supernatants were collected after 48 h of culture and assayed for IL-2 content.

### In Vivo Treatments

Cultured tumor cells were washed three times with PBS and resuspended in PBS for implantation into mice. DBA/2 mice were injected intraperitoneally with 2×10⁵ P815 or 2×10⁴ L1210 tumor cells. Some animals received 3 or 7 injections of 2×10⁶ irradiated P815 tumor cells or hybrid cells, cultured or not with GM-CSF, every 5 days starting on day 3 after, tumor inoculation. In the experiment depicted in FIG. 13, panel B, 2×10⁵ P815 cells were injected intraperitoneally into sublethally irradiated DBA/2 mice (800 rads) and tumor cells harvested from ascites were used to assess tumor resistance in vivo.

### Results

One hybrid displayed morphologic and phenotypic features of dendritic cells and expressed mRNA specific for P815-associated antigen P1A.

2×10⁶ HAT sensitive P815 cells were fused with the same number of bone marrow-derived dendritic cells, as described in Material and Methods. 50 clones proliferated in selection medium containing HAT, and one clone, hybrid 38, displayed morphological features of dendritic cells. As shown in FIG. 9, hybrid cells, cultured with GM-CSF, expressed CD11c, MHC class II and costimulatory molecules (B7-1, B7-2 and HSA). By contrast, P815 mastocytoma cells and hybrid cells cultured in the absence of GM-CSF expressed none of these markers.

Previous publications have shown that the P1A gene is expressed in P815 mastocytoma and encodes a protein that includes a nonapeptide representing a tumor rejection antigen (P815AB; Brichard et al. (1995); Lethé et al. (1995)). Hybrid 38 has been tested for the expression of mRNA specific for P1A and showed that hybrid cells, cultured with or without GM-CSF, as well as P815 tumor cells express mRNA for P1A, whereas DC generated from bone marrow progenitors were negative (FIG. 10). Hybrid 38 is a somatic hybrid (it contains an average of 73 chromosomes) between a dendritic cell, as suggested by the phenotype and function (see below), and a mastocytoma cell, as assessed by expression of mRNA specific for P1A.

Hybrid 38 and bone marrow-generated DC, but not P815, induced primary responses in vitro. Hybrid cells had the capacity to process and present exogenous antigen in the context of class II MHC. FIG. 11 shows that T-cell hybridoma secreted high levels of IL-2 when cultured with GM-CSF treated hybrid cells and insulin protein. No IL-2 was produced in the absence of insulin. Furthermore, since DC appear to have the unique property to activate naive T-cells in vitro, the Inventors have tested the capacity of hybrid cells, P815 and bone-marrow derived DC to induce primary immune responses in vitro. Irradiated, GM-CSF-treated hybrid cells and DC from DBA/2 mice (H-²d) induced proliferation (FIG. 11) and IL-2 secretion (FIG. 11) by purified CD4⁺ T-cells from CBA mice (H-2^{k}). By contrast, P815 and hybrid cells cultured in the absence of GM-CSF did not sensitize allospecific T-lymphocytes in vitro, as assessed by proliferation and IL-2 secretion at background level. Thereafter the role of B7-1 and B7-2 in the induction of primary response was determined. The addition of neutralizing antibodies specific for B7-1 and B7-2 abrogated T-cell proliferation and IL-2 secretion (FIG. 11D). Antibodies to B7-2 alone significantly reduced T-cell activation, whereas anti-B7-1 or isotype- matched control antibodies had no effect.

Repeated injections of hybrid cells prevented the growth of pre-established P815 mastocytoma and induced long-term protection. The potential utility of hybrid-based immunization for the therapy of established tumors was tested in mice inoculated with a lethal dose of P815 intraperitoneally 3 days previously. Mice bearing growing tumor received 7 intraperitoneal injections of 2×10⁶ irradiated (15,000 rads) hybrid cells from day 3 to day 33 after tumor inoculation.

This therapy resulted in long-term tumor protection in 55% (FIG. 12) of the animals. The tumors grew progressively and killed the animals in the control groups that included untreated mice, mice treated with irradiated hybrid cells cultured without GM-CSF, or animals injected with irradiated P815 cells.

The specificity of tumor resistance induced by hybrid cells was demonstrated by the lack of effect of hybrid therapy on the growth of leukemia L1210, a methylcholanthrene-induced leukemia of DBA/2 mice (FIG. 13 panel A). To test whether 7 injections were required to prevent tumor growth, 3 groups of mice were injected with P815, two of them were subsequently treated with irradiated hybrid cells. The data show that 3 or 7 injections of hybrid cells resulted in similar protection (100% and 90%, respectively) to preinjected P815 (FIG. 13 panel A).

Whether hybrid therapy resulted in long-lasting resistance was tested. To avoid the potential helper effect generated by components of the FCS present during culture of hybrid and tumor cells, surviving mice were subsequently injected with P815 cells harvested from irradiated mice inoculated with mastocytoma cells. The data in FIG. 13 (panel B) show that treated mice were protected against a second tumor challenge, whereas all control mice died within 23 days after tumor inoculation.

The tumor resistance induced by hybrid cells correlates with the development of IL-2 secreting cells and tumor-specific cytotoxic T-lymphocytes. To characterize the anti-tumor immunity induced by hybrid cells, splenocytes and peritoneal exudate cells from resistant mice (inoculated with P815, treated with irradiated hybrid cells and challenged with live P815 harvested from ascites, see FIG. 13B) were restimulated in culture with irradiated tumor cells The data in FIG. 14 show that injection of hybrid cells, cultured with GM-CSF, promoted the generation of cells displaying cytotoxic activity to P815 (panel A), as well as the development of IL-2 secreting cells in the spleen (panel B) and in the peritoneal cavity (panel C). These immune responses were dependent on the in vitro restimulation with irradiated P815 cells. No such immune response was detected in untreated mice.

A cancer therapy based on the elimination of tumor cells in vivo by the immune system offers several advantages which include antigen specificity, lack of toxicity, ubiquity and immunological memory which should ensure long-term resistance. The approach to improve the tumor-specific immune response is based on the two-signal theory which implies that two distinct signals are required for optimal activation of T-lymphocytes (Schwartz (1990, Thompson et al. (1995)). The APCs have therefore a dual function and provide the ligands for the T-cell receptor as well as for the CD28 receptor. Since most tumor cells do express specific antigens (recently reviewed by Van den Eynde and van der bruggen (1997)) but do not provide the second signal, it was hypothesized that a limiting factor in the tumor-specific immunity could be a defective antigen presentation due to the lack of costimulation. This hypothesis is strengthened by recent studies from Huang et al. (1994) showing that the priming of an immune response against an MHC class I restricted antigen that is expressed in non-hematopoietic cells, such as a tumor antigen, involves the transfer of that antigen to a host bone marrow-derived cell before its presentation to CD8⁺ T-cells.

Two main approaches have been undertaken to circumvent this defect:

(i) DC have been loaded with tumor antigens in the form of proteins, peptides or unfractionated acid eluted peptides and

(ii) tumor cells have been transduced with genes encoding helper factors or costimulatory molecules (for review, see Young and Inaba (1996)).

In particular, immunization with irradiated P815 transfected with B7-1 gene successfully induced CTL activity in 100% of mice and protected against tumor challenge (Gajewski et al. (1996)). DC pulsed with P815AB alone did not induce T-cell reactivity, whereas the addition of helper peptides led to efficient priming, suggesting that the failure of P815AB to initiate CD8⁺ cell reactivity may be due to defective recruitment of helper T-cells to the afferent phase of the response (Grohmann et al. (1995), Bianchi et al. (1996)).

The present invention shows that somatic hybrid cells formed between tumor cells and DC have unexpectedly the capacity to provide both antigenic and costimulatory signals to T-cells and to induce specific protection against the established parental tumor. P815 mastocytoma has been shown to express five distinct antigens (A, B, C, D, E) recognized by syngeneic cytolytic lymphocytes (bricahrd et al. (1995)). Two of these tumor rejection antigens, P815A and P815B, are encoded by gene P1A and are presented by class I molecule Ld (Van den Eynde et al. (1991)) both of which are expressed by hybrid 38. There is evidence that the antigen P815A/B is of critical importance in the rejection of the tumor, as P815 A and/or B are lost by tumor cells that escape tumor rejection in vivo (Lethé et al. (1992), Brichard et al. (1995)), although antigens CDE are also involved in tumor resistance.

The Inventors have discovered that hybrid cells, but not P815, may express tumor-associated antigens in the context of class II, thereby leading to activation of CD4⁺ cells, whereas both cell populations would express P815-derived peptides in the context of class I MHC hybrid cells and sensitize CD8⁺ cells. Furthermore, hybrid cells, but not the parental tumor, express B7 and HSA molecules, both of which have been shown to provide the costimulatory signal required for optimal activation of T-lymphocytes. Liu et al. (1997) suggest the induction of memory T-cells requires costimulation by either B7 or HSA, while the induction of effector T-cells depends on B7 but not HSA. The characterization of the spontaneous immune response to P815 in a syngeneic host highlights the critical role of B7-CD28 interaction in initiating an antitumor response. An immune response to tumors which do not express B7 is dependent on costimulation by B7-1 and B7-2 expressed by host cells (Yang et al. (1997)) and requires migration to B7-expressing-sites, such as lymph nodes or spleens. However, this response is insufficient to inhibit subsequent outgrowth of tumor unless the response is further strengthened e.g. by sensitization against B7⁺ tumor cells. Of note, inhibition of T-cell migration into lymph nodes eliminates the immune response to the B7⁻, but not to the B7⁺ P815 implanted in the hind footpads of mice (Yang et al. (1997)). The spontaneous immune response to tumor of non-hematopoietic origin may therefore depend on trans-costimulation, whereas unexpectedly injection of hybrid cells would give rise to higher immune response (by cis-costimulation) and allow initiation of the response at the site of the tumor.

The effector cells that mediate the elimination of P815 in vivo most probably involve cytotoxic T-lymphocytes, as well as IL-2 and IFN-γ secreting cells. The tumor resistance induced by hybrid cells correlates with the development of cytotoxic T-lymphocytes in spleen (FIG. 14) as well as IL-2 (FIG. 14) and IFN-γ secreting cells in spleen and at the site of the tumor. More recently, the incidence of a high IFN-γ producing phenotype in draining lymph nodes of mice has been shown to correlate with the frequency of rejection of P815 implanted in the hind footpads (47). Although the same report has underlined the role of IL-12 in rejection of P815 in vivo, no expression of mRNA coding for IL-12 by hybrid cells has been detected.

An efficient immune response may not only prevent tumor growth in vivo, but also limit the onset of antigenic or MHC-loss variants as well as the mechanisms of suppression by the tumor itself.

The immunostimulatory properties of hybrid cells are GM-CSF-dependent, as hybrid cells cultured without GM-CSF do not express MHC class II, B7 nor HSA molecules, do not sensitize naive T-cells in vitro and do not induce tumor resistance in vivo. This observation may be related to the maturation process that is the hallmark of cells from the dendritic family. Langerhans cells and dendritic cells have a specialization of function over time and undergo phenotypic and functional changes during a phenomenon of maturation that occurs spontaneously in vitro (Inaba et al. (1994)) and may be induced in vivo (De Smedt et al. (1996)). Although the factors that induce this process are largely unknown, GM-CSF seems to be involved. Experiments are under way to transfect the gene coding for GM-CSF in hybrid cells and to test their function in vitro and in vivo. Hybrid cell immunization mediates a specific anti-tumor immunity, since no protection was observed against L1210 lymphoma cells, indicating that carry-over of GM-CSF is not the factor inducing tumor rejection.

There is evidence that the CD28 costimulatory pathway is functional in NK cells and plays an important role in their proliferation and cytokine production (Geldhof et al. (1995)). Of note is that hybrid cells, but not P815 cells, are LAK-sensitive targets, suggesting that Hybrid 38 may induce or enhance NK activity. In addition, NK cells are known to be potent producers of IFN-γ at an early stage of activation, and may direct the development of a tumor-specific Thl and CTL response. The in vivo depletion of NK cells prior to immunization with melanoma cells has been shown to abrogate the capacity of spleen cells to generate CD8⁺ tumor specific CTL after in vitro restimulation (Kurosawa et al. (1995)). Therefore, innate (NK) and adaptative (CTL) cytotoxic immune responses appear to be crossregulated and injection of B7⁺ hybrid cells may lead to enhancement of both responses (Kos and Engleman (1996)).

Bone marrow-derived DC have been shown to combine the high T-cell stimulatory properties with the capacity to process and present native antigens (Garrigan et al. (1996)). Fusion experiments have been performed using P815 and dendritic cells isolated from spleen. The yield of hybrid clones was very low, as compared to fusions between P815 and bone marrow-derived DC, and none of them displayed phenotypic and functional features of dendritic cells, suggesting that fusion partners should be proliferating cells or dendritic cells at a more immature stage.

The resulting hybrid cells were shown to induce hepatoma-specific immunity and to protect against intrahepatically implanted small fragments of hepatoma cells when injected, unirradiated, in syngeneic rats.

### Example 13

CD8α⁺, but not CD8α⁻, dendritic cells sensitize T helper-1 type cells in vivo

Since their discovery in 1973, dendritic cells have gained increasing interest from immunologists, since they appear to be the adjuvant of the immune system in vivo. DC are motile and efficiently cluster with T cells, are widely distributed in tissues, carry antigens that are administered intradermally and intravenously, and circulate through lymph and blood probably in route to lymphoid organs (for review, see Steinman, R. M., Pack, M. and K. Inaba. 1997. Immunological Reviews, 156:25-37).

A new population of dendritic cells has been recently discovered that appears to display opposite properties in vitro, murine dendritic cells consist of both conventional CD8α⁻ and CD8α⁺ cells. CD8α⁺ DC appear to express FasL, and through activation with Fas on activated T cells induce their death by apoptosis in vitro (Vremec, D., M. Zorbas, R. Scollay, D. J. Saunders, C. F. Ardavin, L. Wu and K. Shortman, 1992. J. Exp. Med. 176:47-58; Süss G. and K. Shortman, 1996, J. Exp. Med. 183:1789-1796). The CD8α⁺ population resembles the population of dendritic cells in the thymus that plays a role in negative selection of thymocytes.

We have shown previously (Sornasse, T., V. Flamand, G. De Becker, H. Bazin, F. Tielemans, K. Thielemans, J. Urbain, 0. Leo and M. Moser, 1992. J. Exp. Med. 175:15-21; De Smedt, T., M. Van Mechelen, G. De Becker, J. Urbain, 0. leo and M. Moser, 1997, Eur. J. Immunol. 27:1229-1235) that a single injection of antigen-pulsed splenic DC in syngeneic mice induced the activation of T helper cells of type 1 (secreting interferon-γ and IL-2) and type 2 (producing IL-4, IL-5 and IL-10). More recently, we compared the nature of the immune response induced in recipients injected with antigen-pulsed CD8α⁻ or CD8α⁺ dendritic cells.

Both subsets of dendritic cells were purified as follows: mild collagenase (CLSIII; Worthington Biochemical Corp., Freehold, N.J.) digestion for 25 min at room temperature and EDTA treatment were applied to release DC from murine spleen fragments. Spleen cells were washed in Ca⁺⁺-free HBSS medium containing EDTA and further separated into low and high density fractions on a Nycodenz gradient (Nycomed Pharma AS, Oslo, Norway). Low density cells were cultured during 2 h in RPMI containing 2% HY UltroSER (a serum-free medium supplement purchased from Gibco BRL, Merelbeke, Belgium) and 50 μg/ml of GM-CSF. The non-adherent cells were removed by vigorous pipetting. Adherent cells were cultured overnight in the same medium with or without addition of antigen (keyhole limpet hemocyanin, KLH, 50 μg/ml). Dendritic cells were further separated into CD8α⁺ and CD8α⁻ on a miniMacs column using anti-CD8α-coupled microbeads, according to the manufacturer's recommendations (Miltenyi Biotec GmbH, Bergisch-Gladbach, Germany) and washed in PBS (phosphate buffered saline), 3×10⁵ cells in 50-100 μl were injected into the footpads of syngeneic mice. 5 days later, draining lymph nodes were harvested and unseparated lymph node cells were cultured in 2% HY UltroSER-containing RPMI in the presence of serial dilutions of KLH. The proliferation was measured as thymidine incorporation during the last 12-16 h of the 2-day culture. Culture supernatants were assayed for interleukin-2 after 24 h and for interferon-γ after 96 h of incubation. Culture supernatants were assayed for IL-2 content by a standard ELISA. Interferon-γ was quantitated by two-site ELISA using mAb F1 and Db-1, as previously described (T. De Smedt, et al. 1997. Eur. J. Immunol. 27:1229-1235).

The data in FIG. 15 show that both subsets of dendritic cells, pulsed in vitro with KLH, sensitized antigen-specific T cells in vivo, as assessed by proliferation upon antigen restimulation in culture. Controls included untreated mice (NT) and mice that received unseparated dendritic cells (CD8α^{+/-}). Lymph node cells from untreated mice do not proliferate upon stimulation with KLH in vitro. A similar pattern was observed for interleukin-2 secretion. Interestingly, CD8α⁺, but not CD8α⁻, dendritic cells induced the development of interferon-γ-secreting T cells (Th1 cells) in the same conditions. Lymph node cells from mice injected with unseparated dendritic cells secret intermediate levels of interleukin-2 and interferon -γ. These data suggest that CD8α⁺ dendritic cells strongly sensitive antigen-specific naive T cells and are required for Thl development in vivo.

### REFERENCES

Bianchi, R; et al., J. Immunol. 157, pp. 1589-1597 (1996)

Brichard; V. G. et al., Eur. J. Immunol. 25, pp. 664-671 (1995)

Caux, C. et al., Nature (Lond) 360, pp. 258-261 (1992)

De Smedt, T. et al.; J. Exp. Med. 184, pp. 1413-1424 (1996)

Fallarino, F. et al., J. Immunol. 156, pp. 1095-1100 (1996)

Flamand, V. et al., Int. J. Cancer 45, pp. 757-762 (1990)

Gajewski, T.F. et al., J. Immunol. 156, pp. 2909-2917 (1996)

Garrigan, K. et al., Blood 88, pp. 3508-3510 (1996)

Geldhof, A. B. et al., Cancer Research 55, pp. 2730-2733 (1995)

Grohmann, U. et al., Eur. J. Immunol. 25, pp. 2797-2802 (1995)

Grohmann, U. et al., J. Immunol. 158, pp. 3593-3602 (1997)

Huang, A. Y. C. et al., Science (Wash. D.C.) 264, pp. 961-965 (1994)

Inaba, K. et al., J. Exp. Med. 176 pp. 1693-1702 (1992)

Inaba, K. et al., J. Exp. Med. 180, pp. 1849-1860 (1994)

Kos, F. J., and Engleman, E. G., Immunology Today 17, pp. 174-176 (1996)

Kurosawa, S. et al., Immunology 85, pp. 338-346 (1995)

Lethé, B. et al., Eur. J. Immunol. 22, pp. 2283-2288 (1992)

Liu, Y., and Janeway JR, C. A., Proc. Natl. Acad. Sci. USA 89, pp. 3845-3849 (1992)

Liu, Y. et al., J. Exp. Med. 185, pp. 251-262 (1997)

Naquet, P. et al., J. Immunol. 139, pp. 3955-3963 (1987)

Romani, N. et al., J. Exp. Med. 169, pp. 1169-1178 (1989)

Schwartz, R. H. et al., Science (Wash. D.C.) 248, pp. 1349- 1356 (1990)

Thompson, C. B., Cell 81, pp. 979-982 (1995)

Uyttenhove, C. et al., J. Exp. Med. 157, pp. 1040-1052 (1983)

Van den Eynde B. et al., J. Exp. Med. 173, pp. 1373-1384 (1991)

Van den Eynde, B. and Van der Bruggen, P., Current Opinion in Immunology 9, pp. 684-693 (1997)

Yang, G. et al., J. Immunol. 185, pp. 851-858 (1997)

Young, J. W., and Inaba, K., J. Exp. Med. 183, pp. 7-11 (1996)

Zorina, T. et al., J. Immunother. 16, p. 247 (1994)

What is claimed is:

1. A method for producing an anti-tumor response in a mammalian subject in need of anti-tumor treatment, said method comprising administering to said subject a plurality of dendritic cell/tumor cell hybrids wherein said tumor cell corresponds to the tumor cell in said hybrids.

2. A method for producing an anti-tumor response in a mammalian subject in need of anti-tumor treatment, said method comprising administering to said subject autologous immune cells activated in vitro by the method of claim 1.

3. A method for producing an anti-tumor response in a mammalian subject in need of anti-tumor treatment, said method comprising administering to said subject a dendritic-like cell or dentritic cell/tumor cell hybridoma, wherein said tumor cell corresponds to the tumor cell in said mammalian subject.

4. A method for producing an anti-tumor response in a mammalian subject in need of anti-tumor treatment, said method comprising administering to said subject autologous immune cells activated in vitro by co-cultivating in vitro a dendritic cell/tumor cell hybridoma with immune cells from a mammalian subject in need of anti-tumor treatment.
